(19) United States
(12) Patent Application Publication
(10) Pub. No.: US 2014/0329766 A1
(43) Pub. Date: Nov. 6, 2014

(54) Prophylactic Compositions for Management of Microbial Infections in Patients with Brain Injury

(71) Applicant: UTI LIMITED PARTNERSHIP, Calgary (CA)

(72) Inventors: Connie Hoi Yee Wong, Vermont South (AU); Paul Kubes, Calgary (CA); Steven A. Porcelli, Hartsdale, NY (US)

(21) Appl. No.: 14/361,030

(22) PCT Filed: Nov. 28, 2012
(86) PCT No.: PCT/CA2012/001095

§ 371 Date: May 28, 2014

Related U.S. Application Data
(60) Provisional application No. 61/564,039, filed on Nov. 28, 2011.

(51) Int. Cl. A61K 31/7032; A61K 9/00

(52) U.S. Cl. CPC A61K 31/7032; A61K 9/0019
USPC 514/25; 536/16.8

(57) ABSTRACT

The present disclosure pertains to antimicrobial compositions for intravenous administration to patients who have experienced a serious brain trauma, to reduce the risk of occurrence of post-trauma microbial infections. The antimicrobial compositions comprise an α-galacytosylceramide compound and one or more excipients. The present disclosure also pertains to the use of α-galacytosylceramide compounds for the manufacture of antimicrobial medicaments for intravenous administration to patients with serious brain traumas. The present disclosure also pertains to methods for the prophylactic use of the antimicrobial compositions to reduce the risks of occurrence of post-trauma microbial infections.

### TECHNICAL FIELD

The present disclosure relates to fields of neurology, microbiology and medicine. More particularly, the present disclosure relates to compositions comprising glucosylceramides and their use in therapeutic regimens for prevention and/or treatment of microbial infections in survivors of brain traumas caused by strokes or by severe physical head injuries.

### BACKGROUND

A major cause of death in subjects who initially survived physiological brain traumas resulting from ischemic strokes or from serious physical head traumas, is microbial infections. Such brain traumas are accompanied by severe inflammations, and it has been proposed that immunosuppression is a common physiological post-trauma compensatory response to protect the brain during periods of excessive inflammation. The problem is that such immunosuppression responses make brain-trauma patients highly susceptible to microbial infections. For example, up to 95% of stroke patients have at least one relevant infection-related complication within the first three months after the stroke incident. The incidence of infections in stroke patients is typically two-fold to six-fold greater than in the rest of the hospitalized patient population. Approximately one-third of patients recovering from ischaemic stroke die during hospitalization as a consequence of complications arising from microbial infections. Consequently, stroke patients are typically kept and treated in specialized clinical units where such complications are detected earlier than in general hospital units. However, even in specialized stroke units, stroke-associated infections remain one of the major complications in acute stroke.

Microbial infections are also common in victims of severe physical head trauma. In particular, skull fractures or penetrating wounds can cause tearing of the layers of protective tissues (meninges) that surround the brain. The tears provide easy access sites for bacteria to access the brain and establish infections that could spread to the rest of the nervous system if not detected and treated. However, observance of a fever is the first indication that an infection has occurred in or about the brain, by which time, the infection is usually well-established and remedial treatment must be provided in the form of a crisis intervention therapy.

### SUMMARY

The exemplary embodiments of the present disclosure pertain to compositions for intravenous administration to mammalian subjects who have experienced an ischemic stroke or a traumatic brain injury. The compositions generally comprise a glycosylceramide commingled with suitable excipients. The glucosylceramide is preferably a galactosylceramide, such as exemplified by α-galactosylceramide and variants of α-galactosylceramide such as analogs thereof and derivatives thereof. The compositions may additionally comprise a combination of an α-galactosylceramide with one or more other types of antibiotics.

Some exemplary embodiments of the present disclosure pertain to use of the compositions disclosed herein for administration to mammalian subjects who have suffered a severe brain trauma as a result of an ischemic stroke event, or alternatively, a traumatic physical head injury. In such cases, the first dosages of the compositions should be administered to the subject as soon as possible after the occurrence of the brain trauma, for example concurrent with the stroke event, within one hour of stroke onset or physical trauma, within 2 hours of stroke onset or physical trauma, within 3 hours of stroke onset or physical trauma, within 4 hours of stroke onset or physical trauma, within 6 hours of stroke onset or physical trauma, within 8 hours of stroke onset or physical trauma, within 10 hours of stroke onset or physical trauma, within 12 hours of stroke onset or physical trauma, or within 24 hours of stroke onset or physical trauma. The compositions of the present disclosure may also be used for administration to subjects that have suffered brain trauma as a consequence of occurrence of one or more of thrombolysis, thrombectomy or angioplasty.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-1C are micrograph images summarizing 10-min time-lapse videos of tracks laid down by GFP+ cells in livers from (A) sham-operated control mice, (b) post-ischemic Cxcr6^{gfp/+} mice at 8 h, and (c) post-ischemic Cxcr6^{gfp/+} mice at 24 h;

FIG. 2 is a micrograph showing a representative image of GFP+ cells distributed randomly within liver sinusoids (scale bar=50 μm);

FIGS. 3(A)-3(C) are micrograph images of a representative crawling of a GFP+ cell within a liver sinusoid at (A) time 0, (B) after 5 min, and (C) after 10 min. The red line denotes the crawling route (scale bar=50 μm);

FIGS. 4(A)-4(C) are charts showing the crawling velocities of GFP+ cells in livers of (A) control sham-operated mice, (B) post-ischemic Cxcr6^{gfp/+} mice, and (C) Cxcr6^{gfp/+} mice that underwent 2 h of hindlimb ischemia followed by 24-h reperfusion;

FIGS. 5(A)-5(C) are charts showing the percentages of (A) crawling GFP+ cells, (B) stationary GFP+ cells, and (C) pirouetting GFP+ cells in livers from sham-operated control mice and post-ischemic Cxcr6^{gfp/+} mice;

FIG. 6 are micrographs of a representative pirouetting GFP+ cell showing cell surface ruffling and pseudopod protrusion during 8 mins of recording;

FIG. 7 is a chart showing the numbers of iNKT cells in peripheral blood, livers, spleens, lymph nodes, and thymus from sham-operated control mice and post-ischemic mice over a 24-h post-MCAO period;

FIG. 8 is a chart showing CD69 expression in the hepatic iNKT cells of control sham-operated mice (grey tint), in positive control α-GalCer-treated post-ischemic Cxcr6^{gfp/+} mice 24 h after MCAO (blue line), and in post-ischemic Cxcr6^{gfp/+} mice 24 h after MCAO (red line);

FIG. 9 is a chart showing the percentages of iNKT cells with CD69 expression in the peripheral blood (PBL) and selected organs from control sham-operated mice and in post-ischemic Cxcr6^{gfp/+} mice 24 h after MCAO;

FIGS. 10(A)-10(C) are charts showing the production of (A) IL-10, (B) IFNγ and (C) IL-4 in intracellular hepatic iNKT cells from control sham-operated mice and from post-ischemic Cxcr6^{gfp/+} mice after 8 h of reperfusion;

FIGS. 11(A)-11(B) are charts showing switches in immune programming from T_{H}1-type to T_{H}2-type cytokine IFNγ in (A) wild type mice, and (B) Cd1d^{-/-} mice from sham-operated to immediately after MCAO, 4 h after MCAO, and 8 h after MCAO;

FIGS. 12(A)-12(B) are charts showing switches in immune programming from T_{H}1-type to T_{H}2-type cytokine IL-12p70 in (A) wild type mice, and (B) Cd1d^{-/-} mice from sham-operated to immediately after MCAO, 4 h after MCAO, and 8 h after MCAO;

FIGS. 13(A)-13(B) are charts showing switches in immune programming from T_{H}1-type to T_{H}2-type cytokine IL-10 in (A) wild type mice, and (B) Cd1d^{-/-} mice from sham-operated to immediately after MCAO, 4 h after MCAO, and 8 h after MCAO;

FIGS. 14(A)-14(B) are charts showing switches in immune programming from T_{H}1-type to T_{H}2-type cytokine IL-5 in (A) wild type mice, and (B) Cd1d^{-/-} mice from sham-operated to immediately after MCAO, 4 h after MCAO, and 8 h after MCAO;

FIG. 15 is a chart showing the T_{H}2/T_{H}1 ratios for each of the treatments shown in FIGS. 11-14, were determined by dividing the four normalized T_{H}2 cytokines by the four T_{H}1 cytokines;

FIG. 16(A) is a chart showing the bacterial load in the lungs of control sham-operated wildtype mice and Cd1d^{-/-} mice and post-ischemic wildtype mice and Cd1d^{-/-} mice, 16(B) is a chart showing neutrophil infiltration as measured by MPO activity in the lungs of control sham-operated wildtype mice and Cd1d^{-/-} mice and post-ischemic wildtype mice and Cd1d^{-/-} mice, and 16(C) is a chart showing edema formation in the lungs of control sham-operated wildtype mice and Cd1d^{-/-} mice and post-ischemic wildtype mice and Cd1d^{-/-} mice;

FIGS. 17(A)-17(C) are charts showing the bacterial load in the (A) peripheral blood, (B) liver, and (C) spleen of control sham-operated wildtype mice and Cd1d^{-/-} mice and post-ischemic wildtype mice and Cd1d mice;

FIG. 18 is a chart showing the survival rates of post-ischemic wildtype mice and Cd1d^{-/-} mice treated with or without antibiotics;

FIG. 19 is a chart showing the infarct sizes in post-ischemic wildtype and Cd1d^{-/-} mice at 8 h and 24 h after MCAO;

FIG. 20 is a chart showing the effects of prophylactic administration of antibiotics on the infarct sizes in the post-ischemic wildtype mice and Cd1d^{-/-} mice at 8 h and 24 h after MCAO;

FIGS. 21(A)-21(D) are charts showing the effects of prophylactic administration of antibiotics on bacterial loads in the (A) peripheral blood, (B) lungs, (C) livers, and spleens in the post-ischemic wildtype mice and Cd1d^{-/-} mice at 8 h and 24 h after MCAO;

FIG. 22 is a chart showing the bacterial load in the lungs of post-ischemic mice treated with saline or with recombinant mouse IL-10;

FIG. 23 is a chart showing the percentages of CD3+ T-cells with CD69 expression in the peripheral blood, liver, spleen, lymph node, and thymus cells from control sham-operated wildtype mice, Cd1d^{-/-} mice, post-ischemic wildtype mice, and Cd1d^{-/-} mice;

FIG. 24(A) is a chart showing the percentages of CD4+ T-cells with CD69 expression in the peripheral blood, and 24(B) is a chart showing the percentages of CD8+ T-cells with CD69 expression in the liver from control sham-operated wildtype mice, Cd1d^{-/-} mice, post-ischemic wildtype mice, and Cd1d^{-/-} mice;

FIGS. 25(A)-25(E) are charts showing numbers of leukocytes in the (A) peripheral blood, (B) livers, (C) spleens, (D) thymus, and (E) lymph nodes from control sham-operated wildtype mice, Cd1d^{-/-} mice, post-ischemic wildtype mice, and Cd1d^{-/-} mice;

FIGS. 26(A)-26(E) are charts showing numbers of lymphocytes and granulocytes in the (A) peripheral blood, (B) livers, (C) spleens, and (D) thymus from control sham-operated wildtype mice, Cd1d^{-/-} mice, post-ischemic wildtype mice, and Cd1d^{-/-} mice;

FIGS. 27(A)-27(E) are charts showing numbers of T-cells and B-cells in the (A) peripheral blood, (B) liver, (C) spleen, and (D) thymus from control sham-operated wildtype mice, Cd1d^{-/-} mice, post-ischemic wildtype mice, and Cd1d^{-/-} mice;

FIGS. 28(A)-28(E) are charts showing numbers of leukocytes in the (A) peripheral blood, (B) liver, (C) spleen, (D) thymus, and (D) thymus from control sham-operated wildtype mice and Cd1d^{-/-} mice and post-ischemic wildtype mice and Cd1d^{-/-} mice;

FIG. 29(A) is a chart showing the percentage of crawling GFP+ cells, and 29(B) is a chart showing the percentage of stationary GFP+ cells in the livers of control sham-operated mice and post-ischemic Cxcr6^{gfp/+} mice 24 h after MCAO. The post-ischemic Cxcr6^{gfp/+} mice received prophylactic treatments of one of anti-CD1d, iB4, anti-IL12/18, or apyrase just prior to MCAO;

FIG. 30 is a chart showing the crawling velocities of GFP+ cells within the livers of control sham-operated mice and post-ischemic Cxcr6^{gfp/+} mice 24 h after MCAO. The post-ischemic Cxcr6^{gfp/+} mice received prophylactic treatments of one of anti-CD1d, iB4, anti-IL12/18, or apyrase just prior to MCAO;

FIG. 31(A) is a chart showing the crawling velocities of GFP+ cells within the livers of Cxcr6^{gfp/+} mice following administration of α-GalCer and anti-CD1 d, and 31(B) is a chart showing the crawling velocities of GFP+ cells within the livers of Cxcr6^{gfp/+} mice following recombinant IL-12/18 treatment and IL12/18 blockade;

FIG. 32 is a chart showing the effects of systemic administration of β-adrenergic inhibitors propranolol or 6-OHDA, on the crawling velocities of GFP+ cells within the livers of control sham-operated mice and post-ischemic Cxcr6^{gfp/+} mice 24 h after MCAO;

FIG. 33(A) is a chart showing the percentage of crawling GFP+ cells, and 33(B) is a chart showing the percentage of stationary GFP+ cells in the livers of control sham-operated mice and post-ischemic Cxcr6^{gfp/+} mice 24 h after MCAO. The post-ischemic Cxcr6^{gfp/+} mice received prophylactic treatments of one of propranolol or 6-OHDA;

FIGS. 34(A)-34(B) are micrograph images summarizing 10-min time-lapse videos of tracks laid down by GFP+ cells in livers from post-ischemic Cxcr1^{gfp/+} mice receiving a prophylactic administration of (A) propranolol, or (b) 6-OHDA;

FIG. 35 is a chart showing the effects of prophylactic administration of propranolol or 6-OHDA on post-sinusoidal venule velocity in control sham-operated mice and post-ischemic Cxcr6^{gfp/+} mice;

FIG. 36 is a chart showing the effects of prophylactic administration of propranolol or 6-OHDA on infract size in post-ischemic Cxcr6^{gfp/+} mice;

FIG. 37 is a chart showing the effects of prophylactic administration of propranolol or 6-OHDA on survival of post-ischemic Cxcr6^{gfp/+} mice;

FIG. 38(A) is a chart showing the percentage of crawling GFP+ cells, and 38(B) is a chart showing the percentage of pirouetting GFP+ cells in the livers of Cxcr6^{gfp/+} mice 24 h after superfusion with noradrenalin;

FIG. 39 shows representative micrographs of localized noradrenalin superfusion defining the epicentre, side and distant regions relative to the epicentre of noradrenalin as indicated by CD31 fluorescence (the scale bar in the 4× image is 200 μm; the scale bars in the 10× images are 100 μm);

FIG. 40 is a chart showing the crawling velocity of GFP+ cells within the livers of sham-operated Cxcr^{6gfp/+} mice prior to and after localized noradrenalin superfusion;

FIG. 41(A) is a representative micrograph of isolated control iNKT cells, and 41(B) is a representative micrograph of isolated iNKT cells treated with noradrenalin;

FIG. 42 is a micrograph showing the percentages of non-polarized (non-activated) and polarized (activated) iNKT cells were determined in untreated, noradrenalin and propranolol pretreated, and noradrenalin-treated treatments;

FIG. 43(A) is a chart showing the prophylactic effects of α-GalCer on IFNγ production in peripheral blood, 43(B) is a chart showing the prophylactic effects of α-GalCer on MPO activity in lung tissue, and 43(C) is a chart showing the prophylactic effects of α-GalCer on edema in lungs of post-ischemic mice 24 h after MCAO;

FIGS. 44(A)-44(D) are charts showing the prophylactic effects of α-GalCer, propranolol or 6-OHDA on bacterial loads in (A) peripheral blood, (B) lungs, (C) livers, and (D) spleens of control sham-operated mice and post-ischemic Cxcr6^{gfp/+} mice 24 h after MCAO;

FIGS. 45(A)-45(B) are charts showing the effects of a single prophylactic dose of α-GalCer on (A) infarct volumes and (B) ALT levels in post-ischemic mice;

FIG. 46 is a chart showing bacterial loads in the peripheral blood and lungs of post-ischemic Cd1d^{-/-}” mice treated with propranolol;

FIG. 47 is a chart showing the survival of post-ischemic Cd1d^{-/-} mice treated with propranolol;

FIG. 48 are charts showing the intracellular production of (A) IFNγ and (B) IL-10 in control sham-operated mice and post-ischemic mice treated with propanolol;

FIG. 49 is a chart showing the effects of α-GalCer compounds on the percentages of crawling GFP+ cells in livers from sham-operated control Cxcr6^{gfp/+} mice;

FIG. 50 is a chart showing the effects of a single dosage of α-GalCer compounds administered at 2 h after MCAO, on the bacterial load in peripheral blood from wildtype mice, 24 h after MCAO;

FIG. 51 is a chart showing the effects of a single dosage of α-GalCer compounds administered at 2 h after MCAO, on the bacterial load in lungs from wildtype mice, 24 h after MCAO;

FIG. 52 is a chart showing showing the effects of a single dosage of α-GalCer compounds administered 2 h after MCAO treatment, on the bacterial load in livers from control sham-operated wildtype mice and Cxcr6^{gfp/+} mice, 24 h after MCAO;

FIG. 53 is a chart showing the effects of a single dosage of α-GalCer compounds administered at 2 h after MCAO, on the bacterial load in spleens from wildtype mice, 24 h after MCAO;

FIG. 54 is a chart showing the effects of a single dosage of α-GalCer compounds administered at 2 h, on potential liver damage as predicted by the Alanine Transaminase Activity (ALT) assay, in control sham-operated wildtype mice;

FIG. 55 is a chart showing the effects of a single dosage of α-GalCer compounds administered 2 h after MCAO treatment, on potential liver damage as predicted by the Alanine Transaminase Activity (ALT) assay, in mice at 24 h after MCAO;

FIG. 56(A) is a chart showing a scatter plot for identifying lymphocytes, 56(B) is a chart showing the CD3+ cells that were selected from the lymphocyte population shown in (A), 57(C) is a chart showing the CD3+Tet+(iNKT) cell population from (B) and (57(D) is a chart showing the percentages of iNKT cells expressing CD69+;

FIG. 57 is a chart showing the percentages of iNKT cells with CD69 expression in the peripheral blood (PBL) collected from stroke patients, hospital controls, and healthy controls;

FIGS. 58(A)-58(D) are charts showing systemic production of T_{H}2 cytokines in stroke patients, hospital controls, and healthy controls: (A) IL-4, (B) IL-5, (C) IL-10, and (D) IL-13;

FIGS. 59(A)-59(D) are charts showing systemic production of other cytokines in stroke patients, hospital controls, and healthy controls: (A) IL-17, (B) IL-6, and (C) IL-113;

FIGS. 60(A)-60(D) are charts showing systemic production of T_{H}1 cytokines in stroke patients, hospital controls, and healthy controls: (A) IFNγ, (B) TNF-α, (C) IL-12p40, and (D) IL-12p70;

FIG. 61 is a chart showing the percentages of activated iNKT cells in blood samples collected from patients with brain trauma after 24 h, and from healthy controls;

FIG. 62 is a chart showing the percentages of activated iNKT cells in blood samples collected from patients with various brain injuries at 24 h, and from healthy controls, that are expressing CD3+ or CD3+ and CD69+;

FIG. 63 is a chart showing the changes in the percentages of activated iNKT cells in blood samples collected from patients with various brain injuries and from healthy controls that occurred over a 28-day period; and

FIG. 64(A) is a chart showing activation of iNKT cells in individual brain trauma patients over a 28-day period of time, and 64(B) is a chart showing the distribution of all data points for iNKT cell activation from (A).

### DETAILED DESCRIPTION

Unless otherwise defined, all technical and scientific terms used herein have the same meaning as commonly understood by one of ordinary skill in the art to which this invention belongs. In order that the invention herein described may be fully understood, the following terms and definitions are provided herein.

The word “comprise” or variations such as “comprises” or “comprising” will be understood to imply the inclusion of a stated integer or groups of integers but not the exclusion of any other integer or group of integers.

The term “effective amount” as used herein means an amount effective, at dosages and for periods of time necessary to achieve the desired results (e.g. prophylaxis of microbial infections). Effective amounts of a molecule may vary according to factors such as the disease state, age, sex, weight of the subject. Dosage regimes may be adjusted to provide the optimum therapeutic response. For example, several divided doses may be administered daily or the dose may be proportionally reduced as indicated by the exigencies of the therapeutic situation.

The phrase “treatment of” and”treating” include the amelioration of and/or cessation of a disease, a disorder, or symptoms thereof.

The phrases “prevention of” and “preventing” include the avoidance of the onset of a disease, disorder, or a symptom thereof.

The term “prophyactic administration” as used herein means the administration of any composition to a subject, in the absence of any infection indications, to prevent the occurrence of and/or the spreading of a microbial infection within the subject's body.

The term “ameliorate” as used herein means to improve and/or to make better and/or to make more satisfactory.

The term “antibiotic” as used herein means an organic agent or inorganic agent that is capable of inhibiting microbial growth and/or kills microorganisms.

The term “immmunoregulatory” as used herein means pertaining to the regulation of the immune system, and more specifically, to the control of specific immune responses and interactions between B lymphocytes and/or T lymphocytes and macrophages.

The term “medicament” as used herein means a medicinal composition that promotes recovery from injury or ailment.

The term “subject” as used herein includes all mammalian members of the animal kingdom, and specifically includes humans.

The term “cell” as used herein means a single cell as well as a plurality of cells or a population of cells. Administering an agent to a cell includes both in vitro and in vivo administrations.

The terms “about” or “approximately” as used herein, mean within about 25%, preferably within about 20%, preferably within about 15%, preferably within about 10%, preferably within about 5% of a given value or range.

The term “stroke” as used herein means an occurrence of a disturbance in the supply of blood to a mammalian subject's brain that results in rapid loss of brain functions exemplified by an inability to move one or more limbs and/or an inability to understand or formulate speech and/or loss of sight in one or both eyes. The neurological damage caused by the disturbance in supply of blood to the brain may be temporary or permanent, depending on the severity of the disturbance and on how quickly medical intervention was provided.

The term “ischemic stroke” as used herein means a stroke resulting from a lack of blood flow to the brain caused by a blockage(s) in the vascular system supplying the brain. The blockages may be due to one or more of a thrombosis, an embolism, and/or a systemic hypoperfusion.

The term “hemorrhagic stroke” as used herein means a stroke resulting from a rapid accumulation of blood within or about the brain within the skull whereby increased pressure within the skull interferes with blood flow to and through the brain.

The term “traumatic brain injury” as used herein means a severe trauma administered to the brain by an external physical force exemplified by severe impacts, sudden acceleration, sudden de-acceleration, and the like. Common secondary injuries directly related to traumatic brain injury include: (i) alterations or disturbances in the flow of blood to and through the brain resulting in the occurrences of one or more ischemic stroke events, and (ii) significant increases of pressure within the skull resulting in the occurence of hemorrhagic stroke.

The term “golden hour” as used herein means the time period, commonly referred to by emergency medical personnel, that lasts from a few minutes to several hours following a traumatic injury being sustained by a subject, during which there is the highest likelihood that prompt medical treatment will prevent death.

The term “cytokines” as used herein means small cell-signaling protein molecules that are secreted by numerous mammalian cells and are a category of signaling molecules used extensively in intercellular communication primarily for regulating cellular activities. In particular, “cytokines” as used herein refers specifically to immunomodulating agents exemplified by such as interleukins and interferons.

The terms “modulate” and “modulation” as used herein mean that a given function has been changed. For example, the phrase “a complex modulates the activity or activation of NKT cells” means that the complex causes the activity of NKT cells, for example, affect the production of cytokines to be different from what the production would have been in the absence of the complex. The alteration in activity can be, for example, an increase in the amount of cytokines produced in the presence of the complex compared to the amount of cytokines produced in the absence of the complex (activation or inducing of the NKT cell), or a decrease in the amount of cytokines produced in the presence of the complex compared to the amount of cytokines produced in the absence of the complex (suppression of the NKT cell), or a change in the ratio of different cytokines that are produced by the NKT cells.

The term “NKT cells” as used herein means a subset of T cells that expressed the natural killer (NK) cell-associated marker NK1.1 (CD161). Specifically, the term “NKT cells” refers to CD1d-restricted T cells that are present in mice and humans. Upon activation, NKT cells are able to produce large quantities of interferon-gamma (IFNγ), IL-4, and granulocyte-macrophage colony-stimulating factor, as well as multiple other cytokines and chemokines exemplified by IL-2, IL-13, IL-17, IL-21 and TNF-α. The clinical potential of NKT cells lies in the rapid release of cytokines such as exemplified by IL-2, IFN-γ, TNF-α, and IL-4, that promote or suppress different immune responses.

The term “iNKT cells” as used herein means a subset of CD 1 d-dependent NKT cells that express an invariant T cell receptor α (TCR-α) chain, and therefore, are referred to as type I or invariant NKT cells (iNKT) cells. These cells are conserved between humans and mice and are implicated in many immunological processes.

The term “T cells” as used herein means lymphocytes that are characterized by the presence of a T cell receptor (TCR) on the cell surface. They are called “T cells” because they mature in the thymus.

Many patients develop infections shortly after an acute stroke event regardless of optimal management of their clinical recovery environments. Mortality is higher in these patients and the severity of stroke is the strongest determinant of the infectious risk. However, it is controversial whether infections promote neurological worsening or alternatively, represent a marker for severity of disease. The brain and the immune system are functionally linked through neural and humoral pathways, and decreased immune competence with higher incidence of infections has been demonstrated in several acute neurological conditions. In brain ischemia, infections are associated with the activation of the autonomous nervous system and neuroendocrine pathways, which increase the strength of anti-inflammatory signals. Patients who have experienced severe physical head injuries are also very susceptible to microbial infections, more so than the general hospital patient populations. Appearance of infections in patients with acute stroke or with severe head injuries, relies in part on immunological mechanisms triggered by acute brain injury. An excessive anti-inflammatory response is a key facilitating factor for the development of infection, and it is likely that this immunological response represents an adaptive mechanism to brain ischemia.

NKT cells seem to be essential for several aspects of immunity because their dysfunction or deficiency has been shown to lead to the development of autoimmune diseases (such as diabetes or atherosclerosis) and cancers. The clinical potential of NKT cells lies in their ability, when activated, to rapidly produce and release cytokines, such as IL-2, IFN-γ, TNF-α, IL4, IL-13, IL-17, and IL-21, that promote or suppress different types of immune responses. NKT cells recognize pathogen-derived or self-derived glycolipid antigens presented by the major histocompatibility complex (MHC) class I-like protein, CD1d. This is in contrast to conventional T cells, which are activated by peptide antigens presented by MHC class I or II. CD1d, like other CD1 family members, evolved to present lipids to T cells. However, the nature and the source of the various lipids that bind naturally to CD1d remain poorly elucidated.

IFN-γ is a dimerized soluble cytokine that is the only member of the type II class of interferon, and has physiologically important roles in promoting innate and adaptive immune responses. The absence of IFN-γ production or cellular responsiveness in humans and experimental animals significantly predisposes the host to microbial infection. Aberrant IFN-γ expression is associated with a number of autoinflammatory and autoimmune diseases. The importance of IFN-γ in the immune system stems in part from its ability to inhibit viral replication directly, and most importantly from its immunostimulatory and immunomodulatory effects. IFN-γ is produced predominantly by NKT cells as part of the innate immune response. Once antigen-specific immunity has established, IFN-γ is additionally produced by CD4 T_{H}1 and CD8 cytotoxic T lymphocyte (CTL) effector T cells. IFN-γ is the primary cytokine which defines Th1 cells. T_{H}1 cells secrete IFN-γ, which in turn causes more undifferentiated CD4+ cells (T_{H}0 cells) to differentiate into T_{H}1 cells, representing a positive feedback loop s suppressing T_{H}2 cell differentiation.

We have discovered that the occurrence of a severe brain trauma event, for example as a consequence of an ischemic stroke event or a severe physical injury to the head, results in a rapid decrease in the production of IFN-γ by NKT cells. Furthermore, we have surprisingly discovered that a prophylactic administration of an α-galactosylceramide (α-GalCer) composition as soon as possible after the occurrence of a brain trauma event, significantly reduces the occurrence of post-trauma microbial infections. For example, within about 2 hours, about 4 hours, about 6 hours, about 8 hours, about 12 hours, about 16 hours, about 20 hours, about 24 hours, about 36 hours, about 48 hours, about 60 hours, about 72 hours, about 96 hours. We noted and disclose herein that such administration of an α-GalCer composition maintains the production of IFN-γ by NKT cells after the occurrence of brain trauma events. We also have discovered that administration of an α-GalCer composition shortly after a brain trauma event, significantly reduces the occurrence of post-trauma microbial infections. We noted and disclose herein that such post-brain-trauma administration of an α-GalCer composition restores the production of IFN-γ by NKT cells.

Accordingly, some exemplary embodiments of the present disclosure pertain to compositions comprising an α-GalCer compound and/or an α-GalCer derivative and/or an α-GalCer analog for intravenous delivery into subjects who have suffered brain trauma as a consequence of a stroke or of a serious physical injury to the head, particularly when the α-GalCer composition is applied as soon as possible after the occurrence of the brain trauma, e.g., during the “golden hour”.

Some exemplary embodiments of the present disclosure pertain to use of one or more α-GalCer compound and/or α-GalCer derivative and/or α-GalCer analogs to prepare medicaments for intravenous administration into subjects who have suffered brain trauma as a consequence of a stroke or of a serious physical injury to the head.

Some exemplary embodiments pertain to use of the α-GalCer compositions of the present disclosure for prophylactic management, i.e., prevention and/or reducing the risk of occurrence of microbial infections in subjects after they have suffered a serious brain trauma as exemplified by an ischemic stroke event, a hemorrhagic stroke event, a serious physical head injury, and the like.

α-GalCer is an agelasphin derivative developed by Kirin Brewery Co., Ltd., and was given the common name of “KRN7000”. The α-GalCer structure consists of a galactose combined with a ceramide in an alpha-configuration, and is shown as Formula 1 in Table 1. α-GalCer is a specific ligand for human and mouse natural killer T (NKT) cells and exhibits potent anti-tumor activity with murine in vivo experiments including subcutaneous implanted model and metastatic models in the liver and lung. In liver metastatic models, treatment with KRN7000 suppressed the growth of tumors and prolonged the survival term of the tumor-bearing mice.

In addition to α-GalCer, also suitable for manufacture of the antimicrobial α-GalCer compositions of the present disclosure are α-GalCer analogs and α-GalCer derivatives exemplified by the compounds having chemical structures shown in Formulae 2-11 in Table 1.

### Antibiotic Compositions

The immunoregulatory antibiotic compositions of the present disclosure comprise at least one α-GalCer compound exemplified by the chemical structures shown in Formulae 1-11 (Table 1), commingled with one or more pharmaceutically acceptable or pharmacologically acceptable excipients and/or carriers. The antibiotic compositions are preferably formulated for parenteral administration, more particularly for intravenous administration.

TABLE 1

| F# | Code# | Structure | MW |
|---|---|---|---|
| 1. | α-GalCer | 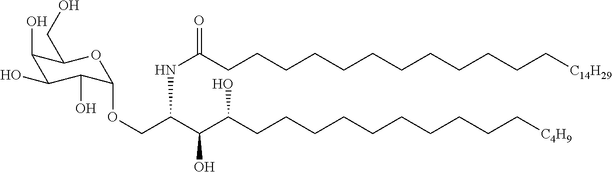 | 853.34 |
| 2. | RB10-1 | 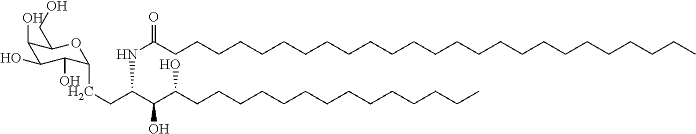 [SMILES: CCCCCCCCCCCCCCCCCCCCCCCCCC(=O)N[C@@H](CCC1OC(CO)C(O)C(O)C1O)[C@H](O)[C@H](O)CCCCCCCCCCCCCC] | 770.13 |
| 3. | DB11-11 | 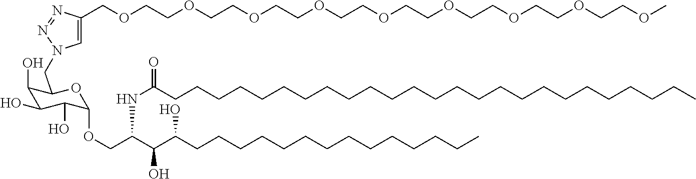 [SMILES: CCCCCCCCCCCCCCCCCCCCCCCCCC(=O)N[C@@H](COC1OC(Cn2cc(COCCOCCOCCOCCOCCOCCOCCOCCOC)nn2)C(O)C(O)C1O)[C@H](O)[C@H](O)CCCCCCCCCCCCCC] | 1305.85 |
| 4. | DB12-1 | 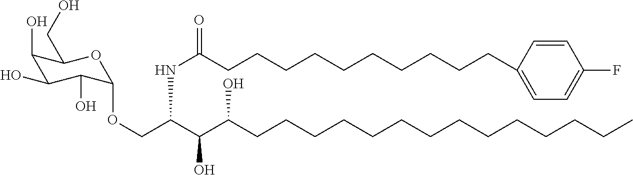 [SMILES: CCCCCCCCCCCCCC[C@@H](O)[C@@H](O)[C@H](COC1OC(CO)C(O)C(O)C1O)NC(=O)CCCCCCCCCCc1ccc(F)cc1] | 742.01 |
| 5. | SKC08- 19 | 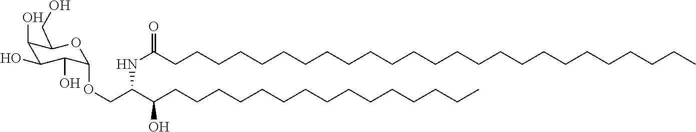 [SMILES: CCCCCCCCCCCCCCCCCCCCCCCCCC(=O)N[C@@H](COC1OC(CO)C(O)C(O)C1O)[C@H](O)CCCCCCCCCCCCCCC] | 842.32 |
| 6. | SKC08- 22a | 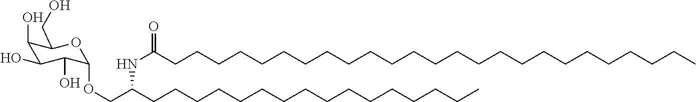 [SMILES: CCCCCCCCCCCCCCCCCCCCCCCCCC(=O)N[C@H](CCCCCCCCCCCCCCCC)COC1OC(CO)C(O)C(O)C1O] | 826.32 |
| 7. | SKC08- 22b | 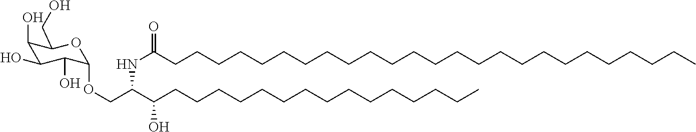 [SMILES: CCCCCCCCCCCCCCCCCCCCCCCCCC(=O)N[C@@H](COC1OC(CO)C(O)C(O)C1O)[C@@H](O)CCCCCCCCCCCCCCC] | 826.32 |
| 8. | SKC08- 25 | 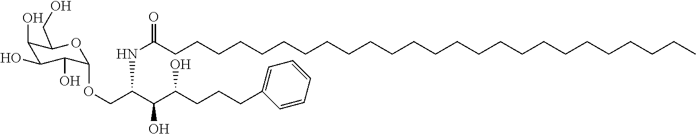 [SMILES: CCCCCCCCCCCCCCCCCCCCCCCCCC(=O)N[C@@H](COC1OC(CO)C(O)C(O)C1O)[C@H](O)[C@H](O)CCCc1ccccc1] | 778.15 |
| 9. | SKC08- 27 | 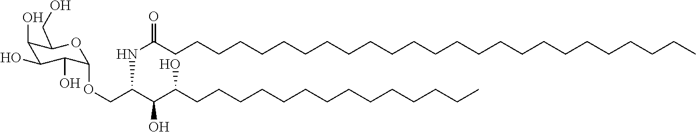 [SMILES: CCCCCCCCCCCCCCCCCCCCCCCCCC(=O)N[C@@H](COC1OC(CO)C(O)C(O)C1O)[C@H](O)[C@H](O)CCCCCCCCCCCCCC] | 856.35 |
| 10. | SKC08- 29 | 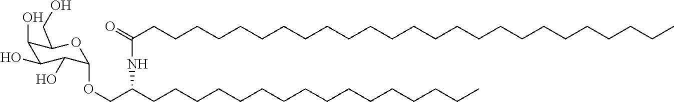 [SMILES: CCCCCCCCCCCCCCCCCCCCCCCCCC(=O)N[C@H](CCCCCCCCCCCCCCCC)COC1OC(CO)C(O)C(O)C1O] | 824.35 |
| 11. | SKC08- 30 | 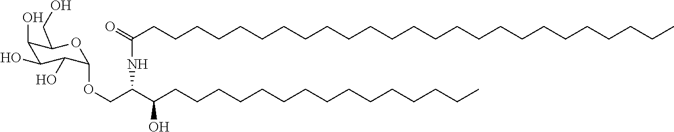 [SMILES: CCCCCCCCCCCCCCCCCCCCCCCCCC(=O)N[C@@H](COC1OC(CO)C(O)C(O)C1O)[C@H](O)CCCCCCCCCCCCCCC] | 840.4 |

F# = Formula number
MW = molecular weight

The phrases “pharmaceutical or pharmacologically acceptable” as used herein refer to compositions that do not produce an adverse, allergic or other untoward reaction when administered to mammalian subject, particularly a human subject. Those skilled in these arts will understand how to prepare the compositions disclosed herein by reference to methods exemplified in “Remington The Science and Practice of Pharmacy. 20th Ed.” (2000, Philadelphia College of Pharmacy and Science). Moreover, it is to be understood that such compositions should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives exemplified by antibacterial agents, antifungal agents, antimivcrobial compounds and the like, isotonic agents, absorption delaying agents, salts, preservatives, drugs, drug stabilizers, gels, binders, excipients, disintegration agents, lubricants, such like materials and combinations thereof, as would be known to one of ordinary skill in the art.

The pharmaceutical compositions of the present invention can easily be administered parenterally by routes exemplified by intravenous administration, intramuscular injection, intrathecal injection, and subcutaneous injection. Parenteral administration can be accomplished by incorporating the compositions of the present disclosure into a solution or suspension. Such solutions or suspensions may also include sterile diluents such as water for injection, saline solution, fixed oils, polyethylene glycols, glycerine, propylene glycol or other synthetic solvents. Parenteral formulations may also include antibacterial agents such as exemplified by benzyl alcohol, methyl parabens and the like, antioxidants such as exemplified by ascorbic acid, sodium bisulfite and the like, and chelating agents exemplified by EDTA. Buffers such as exemplified by acetate buffers, citrate buffers, phosphate buffers and the like, may also be added. Agents for the adjustment of tonicity such as exemplified by sodium chloride and dextrose may also be added. The parenteral compositions may be dispensed into and contained in ampules, disposable syringes or multiple dose vials made of glass or plastic.

The antibiotic compositions of the present disclosure may be formulated with different types of carriers depending on whether it is to be administered in solid, liquid or aerosol form, and whether it needs to be sterile for such routes of administration such as injection. The present disclosure can be formulated for administration intravenously, intradermally, intra-arterially, intraperitoneally, intralesionally, intracranially, intraarticularly, intraprostaticaly, intrapleurally, intratracheally, intranasally, intravitreally, intravaginally, intrarectally, topically, intramuscularly, intraperitoneally, subcutaneously, subconjunctival, intravesicularlly, mucosally, intrapericardially, intraumbilically, intraocularally, orally, topically, locally, inhalation (e.g., aerosol), injection, infusion, continuous infusion, via a catheter, via a lavage, in lipid compositions (e.g., liposomes), or by other method or any combination of the forgoing as would be known to one of ordinary skill in the art.

The actual dosage amount of an antibiotic composition of the present disclosure for parenteral administration to subject can be determined by physical and physiological factors such as body weight, severity of condition, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, the antibiotic compositions may comprise, for example, at least about 0.1% of an α-GalCer compound. In other embodiments, the α-GalCer compound may comprise between about 2% to about 75% of the weight of the unit, or between about 25% to about 60%, for example, and any range derivable therein. In other examples, a dose may also comprise from about 1 μg/kg body weight, about 5 μg/kg body weight, about 10 μg/kg/body weight, about 50 μg/kg body weight, about 100 μg/kg body weight, about 200 μg/kg body weight, about 350 μg/kg body weight, about 500 μg/kg body weight, about 1 mg/kg body weight, about 5 mg/kg body weight, about 10 mg/kg body weight, about 50 mg/kg body weight, about 100 mg/kg body weight, about 200 mg/kg body weight, about 350 mg/kg body weight, about 500 mg/kg body weight, to about 1000 mg/kg body weight or more per administration, and any range derivable therein. Examples of a derivable range from the numbers listed herein, a range of about 5 μg/kg body weight to about 500 mg/kg body weight, about 5 μg/kg body weight to about 250 mg/kg body weight, about 5 μg/kg body weight to about 100 mg/kg body weight, about 5 μg/kg body weight to about 50 mg/kg body weight, about 5 μg/kg body weight to about 25 mg/kg body weight, about 5 μg/kg body weight to about 10 mg/kg body weight, and therebetween.

The pharmaceuticals may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

The carrier can be a solvent or dispersion medium comprising but not limited to water, ethanol, polyols exemplified by glycerol, propylene glycol, liquid polyethylene glycol, and the like, lipids exemplified by triglycerides, vegetable oils, liposomes, and the like, and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as those exemplified by sugars, sodium chloride, and combinations thereof.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The amount of the α-GalCer composition that is effective in the prophylatic treatment or prevention of a microbial infection can be determined by standard clinical techniques. In addition, in vitro or in vivo assays can optionally be employed to help identify optimal dosage ranges. The precise dose to be employed will also depend on the route of administration, and the seriousness of the disorder being treated and should be decided according to the judgment of the practitioner and each subject's circumstances in view of, e.g., published clinical studies. Suitable effective dosage amounts, however, typically range from about 1 μg/kg to about 10,000 μg/kg body weight weekly although they are typically about 1,000 μg/kg or less of body weight on a weekly basis. In one embodiment, the effective dosage amount ranges from about 10 μg/kg to about 5,000 μg/kg of body weight on a weekly basis. In another embodiment, the effective dosage amount ranges from about 50 μg/kg to about 1,000 μg/kg of body weight on a weekly basis. In another embodiment, the effective dosage amount ranges from about 75 μg/kg to about 500 μg/kg of body weight on a weekly basis. The effective dosage amounts described herein refer to total amounts administered, that is, if more than one α-GalCer compound is administered, the effective dosage amounts correspond to the total amount administered. The α-GalCer compositions can be administered as a single daily dose or as divided doses.

### Methods of Use

The present disclosure contemplates, in an exemplary embodiment, the treatment of subjects suffering from or at risk of post-brain-trauma microbial infections. Treatment is defined broadly as attaining any useful clinical endpoint, such as inhibition of growth, reduction of bacterial load, elimination or limitation of bacteria in the lung, increased pulmonary performance, improved patient comfort/stamina, increased patient lifespan, reduction is disease duration, inhibition or prevention of relapse, or enhancement of disease resolution.

Administration of the α-GalCer compositions of the present disclosure will be via any common route so long as the target tissue is available via that route, although intravenous delivery is specifically contemplated. Systemic administration may be achieved by intravenous administration, intradermal injection, subcutaneous injection, intramuscular injection, or intraperitoneal injection. Such compositions would normally be administered as pharmaceutically acceptable compositions. Upon formulation, solutions will be administered in a manner compatible with the dosage formulation and in such amount as is therapeutically effective.

### Kits

The present disclosure also provides kits, such as therapeutic kits. For example, a kit may comprise one or more pharmaceutical composition as described herein and optionally instructions for their use. Kits may also comprise one or more devices for accomplishing administration of such compositions. For example, a subject kit may comprise a pharmaceutical composition and device for accomplishing injection of the pharmaceutical composition, for inhalation therapy, or for catheterization.

Kits may comprise a container with a label. Suitable containers include, for example, bottles, vials, and test tubes. The containers may be formed from a variety of materials such as glass or plastic. The label on the container may indicate that the composition is used for a specific therapy, and may also indicate directions for uses such as those described above. The kit of the disclosure will typically comprise the container described above and one or more other containers comprising materials desirable from a commercial and user standpoint, including buffers, diluents, filters, needles, syringes, and package inserts with instructions for use.

Some exemplary embodiments pertain to emergency response kits for use in providing a prophylactic therapeutic treatment to brain trauma victims to reduce the potential for infection. The kits comprise at least a dosage of an α-GalCer composition of the present disclosure, an instrument exemplified by a syringe for delivering the dosage of α-GalCer to a brain trauma victim, sterilizing materials for surface sterilization of a selected target on the suffice of the victim's skin prior to administration of the dosage of α-GalCer.

### Combined Therapy

In order to increase the effectiveness of the α-GalCer compositions of the present disclosure, it may be desirable to combine these compositions with another antimicrobial agent effective in the treatment or prevention of infections. The terms “contacted” and “exposed,” when applied to a cell, tissue or organism, are used herein to describe the process by which agents of the present disclosure are delivered to a target cell, tissue or organism or are placed in direct juxtaposition with the target cell, tissue or organism. In a particular embodiment, the claimed therapies is utilized with antibiotics in a “one-shot” prevention of brain trauma/stroke-related infection.

The therapy or prophylaxis according to the present disclosure may precede, be co-current with and/or follow the other agent(s). In embodiments where the treatment according to the present disclosure and other agent(s) are applied separately to a cell, tissue or organism, one would generally ensure that a significant period of time did not expire between the time of each delivery, such that the therapy according to the present disclosure and other agent(s) would still be able to exert an advantageously combined effect on the cell, tissue or organism. Various combination regimens of therapy according to the present disclosure and one or more other anti-bacterial agents may be employed. Non-limiting examples of such combinations are shown below, wherein therapy according to the present disclosure is “A” and the other agent or therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of therapy according to the present disclosure to a cell, tissue or organism may follow general protocols for the administration of pharmaceuticals, taking into account the toxicity, if any. It is expected that the treatment cycles would be repeated as necessary. In particular embodiments, it is contemplated that various additional agents may be applied in any combination with the present disclosure.

Antibiotics that may be employed include the aminoglycosides (Amikacin (IV), Gentamycin (IV), Kanamycin, Neomycin, Netilmicin, Paromomycin, Streptomycin (IM), Tobramycin (IV)), the carbapenems (Ertapenem (IV/IM), Imipenem (IV), Meropenem (IV)), Chloramphenicol (IV/PO), the fluoroquinolones (Ciprofloxacin (IV/PO), Gatifloxacin (IV/PO), Gemifloxacin (PO), Grepafloxacin (PO), Lomefloxacin, Moxifloxacin (IV/PO), Norfloxacin, Ofloxacin (IV/PO), Sparfloxacin (PO), Trovafloxacin (IV/PO)), the glycopeptides (Vancomycin (IV), the lincosamides (Clindamycin (IV/PO), Clarithromycin (PO), Dirithromycin, Erythromycin (IV/PO), Telithromycin), the cephalosporins (Cefadroxil (PO), Cefazolin (IV), Cephalexin (PO), Cephalothin, Cephapirin, Cephradine, Cefaclor (PO), Cefamandole (IV), Cefonicid, Cefotetan (IV), Cefoxitin (IV), Cefprozil (PO), Cefuroxime (IV/PO), Loracarbef (PO), Cefdinir (PO), Cefditoren (PO), Cefixime (PO), Cefoperazone (IV), Cefotaxime (IV), Cefpodoxime (PO), Ceftazidime (IV), Ceftibuten (PO), Ceftizoxime (IV), Ceftriaxone (IV), Cefepime (IV)), monobactams (Aztreonam (IV)), nitroimidazoles (Metronidazole (IV/PO)), oxazolidinones (Linezolid (IV/PO)), penicillins (Amoxicillin (PO), Amoxicillin/Clavulanate (PO), Ampicillin (IV/PO), Ampicillin/Sulbactam (IV), Bacampicillin (PO), Carbenicillin (PO), Cloxacillin, Dicloxacillin, Methicillin, Mezlocillin (IV), Nafcillin (IV), Oxacillin (IV), Penicillin G (IV), Penicillin V (PO), Piperacillin (IV), Piperacillin/Tazobactam (IV), Ticarcillin (IV), Ticarcillin/Clavulanate (IV)), streptogramins (Quinupristin/Dalfopristin (IV), sulfonamide/folate antagonists (Sulfamethoxazole/Trimethoprim (IV/PO)), tetracyclines (Demeclocycline, Doxycycline (IV/PO), Minocycline (IV/PO), or Tetracycline (PO)).

The following examples are included to demonstrate preferred embodiments of the disclosure. It should be appreciated by those of skill in the art that the techniques disclose d in the examples which follow represent techniques discovered by the inventors to function well in the practice of the disclosure, and thus can be considered to constitute preferred modes for its practice.

### Example 1

### Materials, Methods, and Treatments

### (A) Mice.

Balb/c and CD 1 d-deficient Balb/c background (Cd1d^{-/-}) mice were purchased from The Jackson Laboratory (Bar Harbour, Me., USA). Cxcr6^{gfp/+} knock-in mice on the Balb/c background were a gift from Dr. Dan R. Littman (New York University School of Medicine, New York, N.Y.). Mice were maintained in a specific pathogen-free, double-barrier unit at the University of Calgary. All protocols used were in accordance with the guidelines drafted by the University of Calgary Animal Care Committee and the Canadian Council on the Use of Laboratory Animals. Male mice of 8-12 weeks old were used in the study.

### (B) Mouse Focal Cerebral Ischemia Model.

Mice underwent the mid-cerebral artery occlusion (MCAO) model of cerebral ischemia-reperfusion injury as previously described (Connolly, Jr. et al., 1996; Wong et al., 2008). Male mice of 8-12 weeks old were anesthetized by intraperitoneal injection of a mixture of 10 mg/kg xylazine hydrochloride (MTC Pharmaceuticals, Cambridge, ON) and 200 mg/kg ketamine hydrochloride (Rogar/STB, London, ON). Body temperature was maintained at 37° C. using a heating pad and temperature regulator with rectal probe. Mice were randomly divided into 2 groups: (i) sham-operated, and (ii) MCAO-operated. All surgical instruments were sterilized before the surgery. Before any incision, the area was swabbed with ethanol. To induce MCAO, a 10-mm incision was made on the right-hand side of the neck. Then, the common carotid artery, external carotid artery, and internal carotid artery were dissected free. The external carotid artery was further dissected distally, then coagulated and cut to serve as a stump. After applying temporary clamps at the common carotid artery and internal carotid artery, a monofilament with a silicon coating diameter of 0.21-0.23 mm (Doccol Corporation, Redlands, Calif., USA) was inserted into the stump of the external carotid artery. The monofilament then advanced a defined distance (12 mm) so that the distal end of the monofilament came to rest across the origin of the MCA. The stump of external carotid artery was tied off. The wound on the neck of the mouse was sutured and the mouse was then transferred onto a heat pad to maintain its body temperature at 37° C. After 30 mins of occlusion, the monofilament was withdrawn to allow reperfusion to occur. The animal then recovered from the anaesthesia. Rehydration of the mice was performed every 4 h with a subcutaneous injection of electrolytes and glucose. Food pellets softened with water were placed in the cage after surgery as the mice recovered, and access to water was provided. Sham-operated animals were subjected to the initial anaesthetic and neck incision only. The animals were then allowed to recover from anaesthetic. All animals were put onto a 37° C. heat pad, post-surgery, to recover from anaesthesia until euthanized.

### (C) Treatments.

To determine the effect of preventive antibacterial treatment, animals received a combination of Gentamicin (4 mg/kg) and Ampicillin (100 mg/kg) subcutaneously three times a day. Administration of the antibiotics started 2 h after MCAO. Control animals were treated with the diluent.

In separate experiments, α-GalCer (2 μg/mouse; KRN-7000; Kirin Brewery, Gunma, Japan; see Formula 1, Table 1) was dissolved in 0.5% Tween 20 plus 0.9% NaCl and injected intravenously into post-ischemic mice 2 h after MCAO.

For experiments with recombinant cytokines, recombinant mouse IL-12 (2 ng/mouse; R&D systems, Minneapolis, Minn.) and IL-18 (200 ng/mouse; Medical & Biological Laboratories Co., Ltd., MBL) was injected intravenously 45 mins prior to imaging. Recombinant IL-10 (1 μg/mouse; Cedarlane, Ontario, Canada) was injected intravenously 1 h prior to MCAO. Anti-mouse CD1d neutralizing antibody (200 μg/mouse; 1B1; eBioscience) or anti-mouse IL-12 (200 μg/mouse; C17.8; eBioscience) and anti-mouse IL-18 (50 μg/mouse; 112624; R&D systems) blocking antibodies were injected intravenously 1 h prior to MCAO. For β-adrenoreceptor blockade, propranolol (Sigma), was dissolved in 0.9% sodium chloride at 3 mg/ml, and administered at 30 mg/kg body weight at 4 and 8 h after MCAO. 6-hydroxydopamine (6-OHDA; Sigma), used to chemically sympathectomize peripheral nerve terminals containing noradrenalin, was injected intraperitoneally at a concentration of 250 mg/kg 3 days prior to MCAO surgery.

### Example 2

### Microscopic Examination of Liver Cells after Induced Stroke Events in Mice

### (A) Intravital Microscopy of the Liver.

Murine liver intravital microscopy was performed as taught by Wong et al. (1997, A minimal role for selectins in the recruitment of leukocytes into the inflamed liver microvasculature. J. Clin. Invest. 99: 2782). Briefly, the tail vein of the anesthetized mice was cannulated to administer fluorescently labeled antibodies and/or additional anesthetic as required. Body temperature was maintained at 37° C. using an infrared heat lamp. Mice were placed in a right lateral position on an adjustable microscope stage. A lateral abdominal incision along the costal margin to the midaxillary line was made to exteriorize the liver, and all exposed tissues were moistened with saline-soaked gauze to prevent dehydration.

### (B) Analysis of Intravital Imaging Videos.

Cells were tracked using ImageJ software version 1.45 (NIH at http://rsb.info.nih.gov/ij/). Time-lapse videos exported from Volocity were imported as .mov file, and then converted to 8-bit grayscale. Fluorescent cells were adjusted using threshold control, and noise particles less than 2.0 pixels were removed by filter. Movement of cells was measured using manual tracking, and was calculated for each track using ImageJ output spreadsheets. The cell velocity was expressed in distance (μm) per time (min). 30-40 tracks from 10 mins videos of each animal in each experimental group were analyzed.

### (C) Spinning Disk Confocal Intravital Microscopy.

Excised livers were prepared for in vivo microscopic observation. Briefly, the liver was placed on the pedestal of an inverted microscope and the liver surface was then covered with a small piece of saline-soaked KimWipe to hold the organ in position. The liver microvasculature was visualized using a spinning-disk confocal microscopy and images were acquired with an Olympus IX81 inverted microscope using a ×10/0.40 UplanFL N objective. The microscope was equipped with a confocal light path (WaveFx, Quorum, Guelph, ON, CA) based on a modified Yokogawa CSU-10 head (Yokogawa Electric Corporation, Tokyo, Japan). Cxcr6^{gfp/+} mice were used to visualize hepatic iNKT cells. Laser excitation wavelengths (Cobolt, Stockholm, Sweden) were used in rapid succession and visualized with the appropriate long-pass filters (Semrock, Rochester, N.Y.). Typical exposure time for each excitation wavelength was 300 milli-seconds. A 512×512 pixel back-thinned electron-multiplying charge-coupled device camera (C9100-13, Hamamatsu, Bridgewater, N.J.) was used for fluorescence detection. Volocity Acquisition software (Improvision) was used to drive the confocal microscope. Auto contrast was used. Images were captured at 16 bits/channel in RGB. Behaviors of iNKT cells in the hepatic microvasculature were assessed.

### (D) Results.

It is known that iNKT cells primarily reside in the liver and spleen. Furthermore, it is known that iNKT cells patrol the hepatic microvasculature and can be tracked in Cxcr6^{gfp/+} mice. When activated with either CDId ligands or exogenous administration of interleukin (IL)-12 and IL-18, iNKT cells show altered behavior, including cessation of intravascular crawling associated with activation and release of key cytokines.

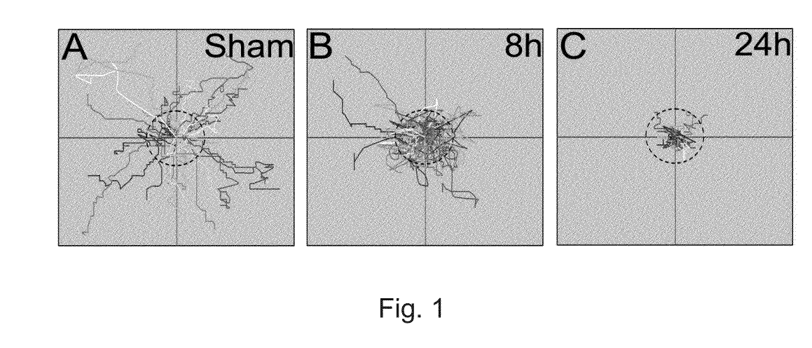

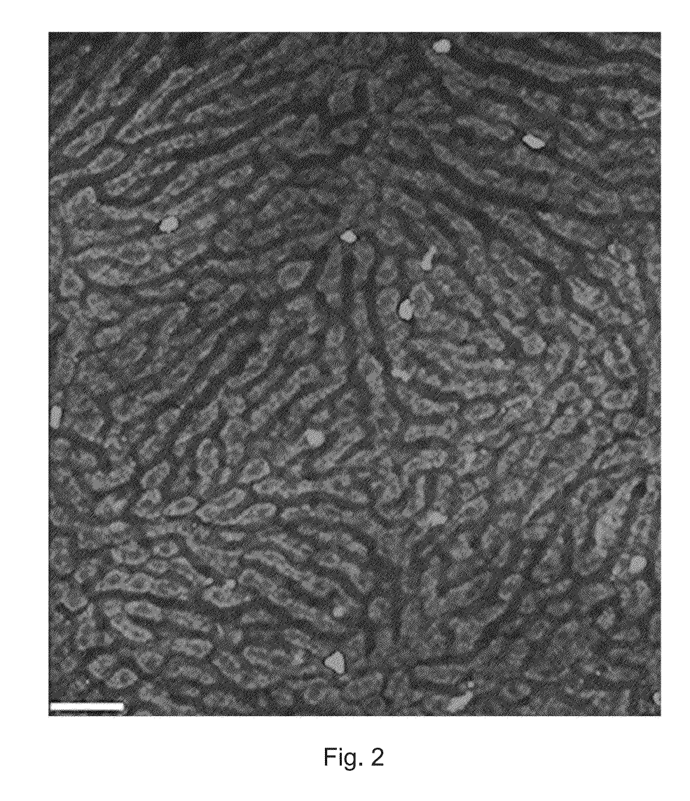

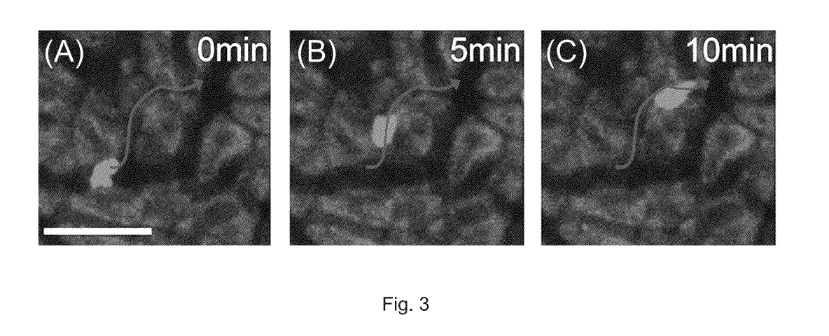

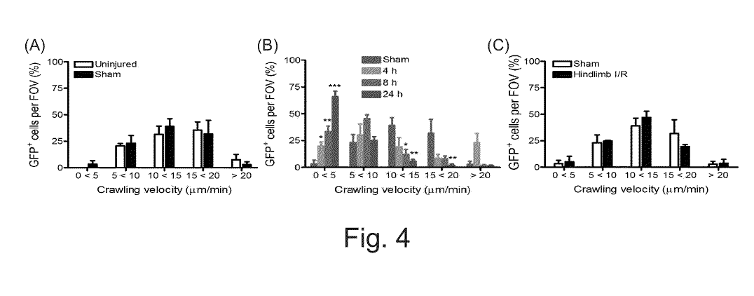

We hypothesized that, on the basis of their intravascular localization, iNKT cells are well-positioned to detect distant tissue injury and participate in systemic immunomodulation. Liver iNKT cell behavior in response to transient midcerebral artery occlusion (MCAO)-induced brain injury was examined in a rodent model of stroke. Intravital spinning disk confocal microscopy observations indicated that under sham i.e., control conditions, iNKT cells crawl randomly within liver sinusoids (FIGS. 1A, 2, 3). FIG. 1A is a micrograph showing the tracks of green fluorescent protein-positive (GFP+) cells within the liver during 10 min of recording. Paths were normalized from their origins, and the dotted circle denotes 10 μm radius from origin (N=4 individual mice). FIG. 2 is a micrograph showing a representative image of GFP+ cells distributed randomly within liver sinusoids (the scale bar indicates 50 μm). FIG. 3 is a micrograph showing a representative crawling GFP+ cell within a liver sinusoid during 10 mins of recording. The red arrow denotes the route of crawling (the scale bar indicates 50 μm). It was noted that the crawling velocities of iNKT cells in control and sham-operated animals did not differ (FIG. 4A; data are expressed as the percentage of GFP+ cells per field of view).

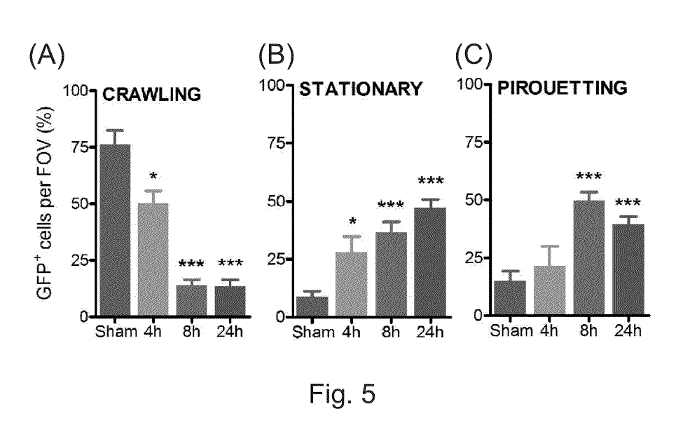

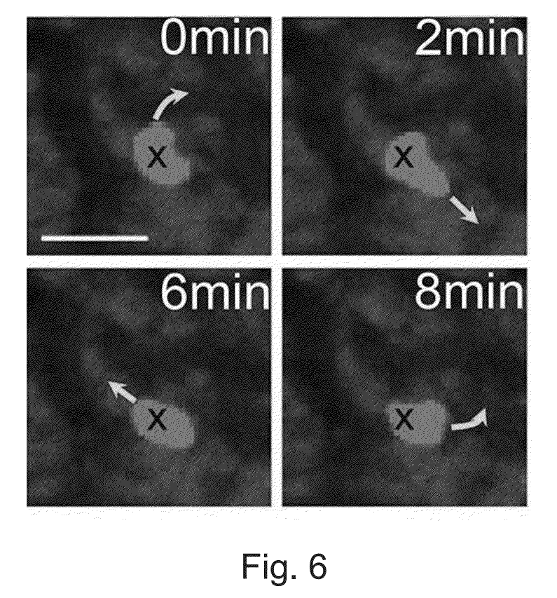

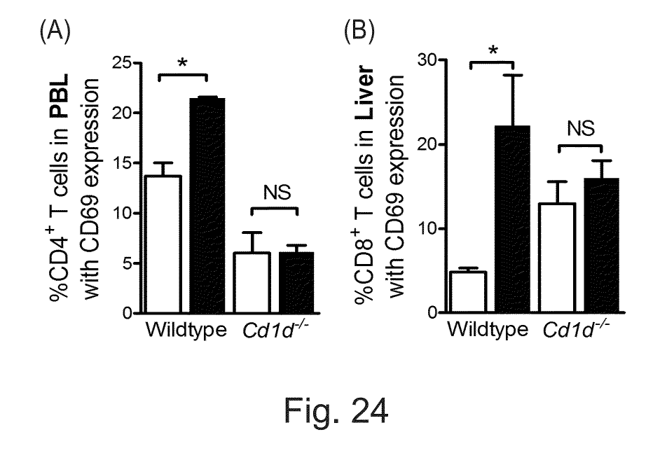

In contrast, markedly restricted crawling of liver iNKT cells was observed as reperfusion progressed after MCAO (FIGS. 1B-1C, 4B). FIGS. 1B and 1C show the tracks of GFP+ cells within the livers from Cxcr6^{gfp/+} mice during 10 min of recording, 8 h after the ischemia procedure (FIG. 1B) and 24 h after MCAO (FIG. 1C). FIGS. 3A-3C are micrographs showing the crawling of a GFP+ cell within a liver sinusoid during 10 mins of recording. The red arrow denotes the route of crawling (the scale bar indicates 50 μm). Significant decreases in the number of crawling iNKT cells and increasing numbera of stationary iNKTcells were observed at 4 h, 8 h, and 24 h after MCAO (FIGS. 5A, 5B). Some of the arrested cells continued to send out pseudopods in “pirouetting” or “scanning” circular patterns (FIGS. 5C, 6). These behaviors were particular to the ischemia-reperfusion in the brain, as ischemia-reperfusion injury of the hindlimb had no effect on the behavior of liver iNKT cells (FIG. 4C).

### Example 3

### Effects of Stroke Events on Expression of iNKTcells

### (A) Flow Cytometric Analysis.

Blood was collected from anaesthetized mice by cardiac puncture, red blood cells were lysed with ACK lysis buffer (Lonza, Switzerland) and leukocytes were washed in cold FACS wash buffer (FWB; PBS, 2% fetal calf serum, 0.5 mM EDTA). Mice were euthanized and organs were collected into cold FWB. Single cell suspensions were generated from liver, spleen, thymus, and peripheral lymph nodes by mechanical disruption through a 40 μm nylon mesh. Liver cell suspensions were washed with cold PBS, resuspended in 44% isotonic Percoll (Sigma) and layered over 70% isotonic Percoll. Gradients were resolved through centrifugation for 20 min at 400×g in a bench-top centrifuge with no brake, after which the interface containing the lymphocytes was collected and washed in FWB.

For enumeration of leukocyte populations, single cell suspensions of peripheral blood, liver, spleen, thymus and lymph nodes from sham-operated and post-ischemic wild-type mice and Cd1d^{-/-} mice were stained with PerCP-conjugated anti-CD45 (30-F11, BD Pharmingen), Alexa Fluor 450-conjugated anti-CD3 (145-2C11, eBioscience), PE-conjugated anti-B220 (RA3-6B2, BD Pharmingen), PE-conjugated anti-CD4 (RM4-4, eBioscience), PE-conjugated anti-NKp46 (29A1.4, eBioscience), PE-conjugated PB57-loaded CD1d-tetramer (NIH, Atlanta, Ga.) and eFluor 605NC-conjugated anti-CD8 (53-6.7, eBioscience).

For analysis of cellular activation single cells suspensions of blood, liver, spleen, thymus and lymph nodes from sham-operated and post-ischemic wild-type and Cd1d^{-/-} were stained with Alexa Fluor 450-conjugated anti-CD3, PE-conjugated anti-NKp46, PE-conjugated PBS57-loaded CD1d-tetramer, eFluor 605NC-conjugated anti-CD8, PE-conjugated anti-CD4 and PerCPconjugated anti-CD69 (H1.2F3, BD Pharmingen).

For intracellular cytokine analysis, liver single cell suspensions from sham-operated and post-ischemic Cxcr6^{gfp/+} mice were stained with PerCP-conjugated anti-CD69, washed with cold FWB, resuspended in Cytofix/Cytoperm (BD Bioscience) for 30 min on ice. Samples were washed in Perm/Wash (BD Bioscience) and then stained with Alexa Fluor 450-conjugated anti-IFNγ (XMG1.2, eBioscience) and PE-conjugated anti-IL-4 (11B11, BD Pharmingen) or PE-conjugated anti-IL-10 (JESS-16E3, eBioscience).

All samples were analysed on an Attune acoustic focusing cytometer (Applied Biosystems).

### (B) Measurement of Cytokine Levels.

Anaesthetized mice from Example 1 were washed with 70% ethanol under sterile conditions. Blood was collected by cardiac puncture and allowed to clot at room temperature for at least 30 mins. The sample was then centrifuged at 400×g for 10 mins for the retrieval of serum. The serum samples were analyzed for inflammation-relevant cytokines and chemokines by using a Luminex 200 apparatus (Applied Cytometry Systems, UK) and a multiplex mouse cytokine/chemokine kit from Millipore (Massachusetts, USA) according to manufacturer′fs instructions. The panel included IFNγ, IL-4, IL-5, IL-6, IL-9, IL-10, IL-12p40, IL-12p70, IL-13 and TNFα. The data were analyzed with the StarStation V.2.3 software from Applied Cytometry Systems (UK).

### (C) Results.

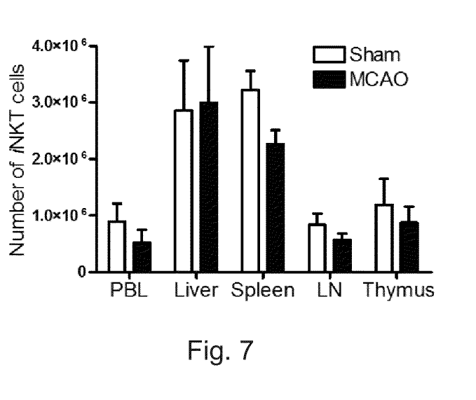

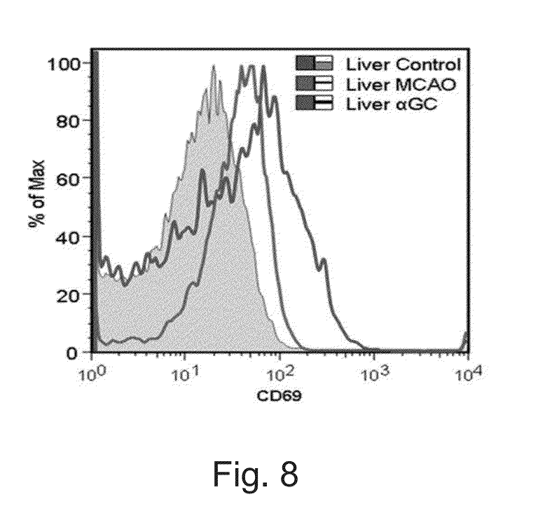

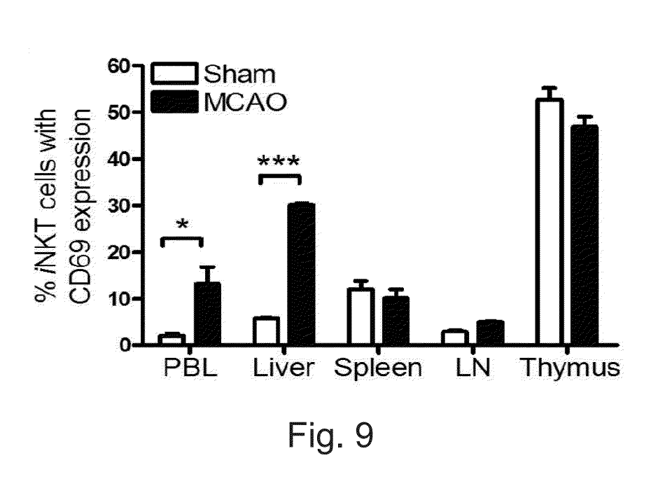

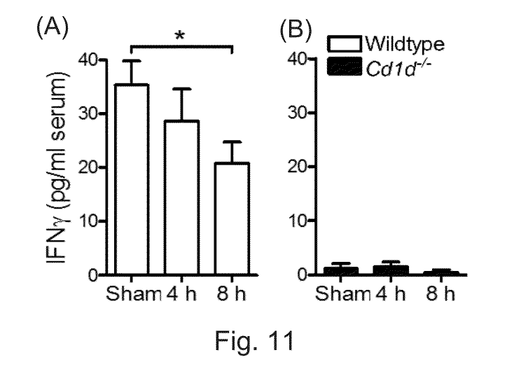

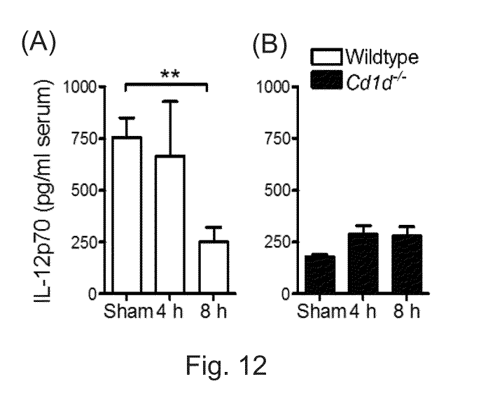

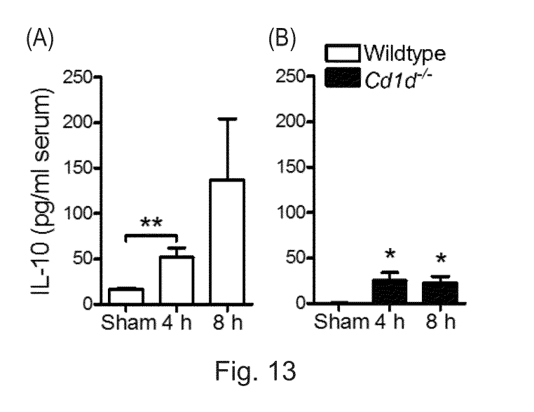

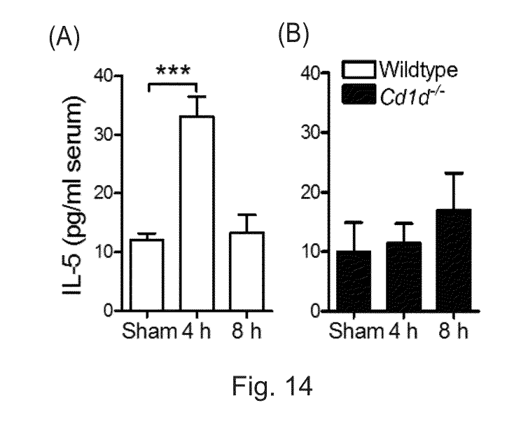

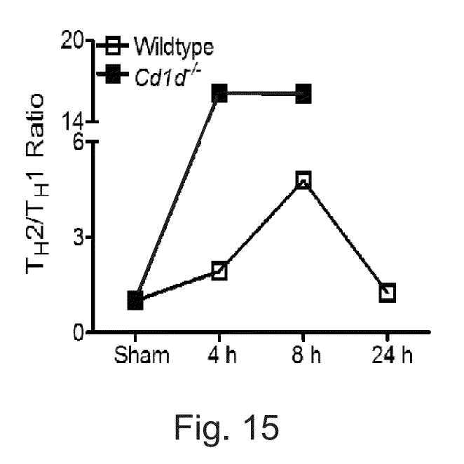

It is known that stroke induces major immune changes, including severe lymphopenia in the peripheral blood, thymus, and spleen. Interestingly, in this study, significant changes in the numbers of iNKT cells in the peripheral blood, liver, spleen, thymus, and lymph node of post-ischemicmice, were not observed (FIG. 7). However, increased expression of CD69 in iNKTcells was observed in the peripheral blood and liver (FIGS. 8, 9), which suggests regional iNKT cell activation occurred after the stroke treatment (FIG. 9). Taken together, these data demonstrate that the effects of brain trauma are far-reaching, and include the capacity to induce profound behavioral changes in hepatic iNKT cells. It is known that activated iNKT cells produce cytokines and chemokines. After MCAO in this study, systemic TH1-type cytokines such as interferon-γ (IFN-γ) and IL-12p70 decreased in wild-type mice (FIGS. 11, 12), reaching significance at 8 h reperfusion. By contrast, T_{H}2-type cytokines, including IL-10 and IL-5, were increased at 4 h after MCAO (FIGS. 13, 14). IL-4 was not detected at any time in this study. In the liver, iNKT cells produced significantly increased amounts of IL-10, but not of IFN-γ or IL-4, at 8 h after MCAO (FIG. 12). The increased ratio of T_{H}2-type over T_{H}1-type cytokines in post-ischemic wild-type mice highlights a general switch in systemic immunity from T_{H}1-type to T_{H}2-type in the early stages of reperfusion after stroke (FIG. 15).

### Example 4

### Effects of Stroke Events on Immunomodulatory Responses and Incidence of Microbial Infections

### (A) Bacteriological Analysis.

The anaesthetized mice from Example 1 were washed with 70% ethanol under sterile conditions. Blood was collected by cardiac puncture. The lungs, liver and spleen were removed after thoracotomy and homogenized. For determination of colony forming units (CFU), 10 μl of tissue homogenate or blood was serially diluted, plated onto brain heart infusion (BHI) agar plates supplemented with 5% sheep blood (Daylnn Biologicals, Alberta, Canada), incubated at 37° C. for 18 h, and bacterial colonies were counted.

### (B) Lung Injury Assessment.

The lungs of anaesthetized mice were removed after specified period of reperfusion. For determination of lung myeloperoxidase (MPO) activity (an indicator of lung inflammation), samples of lungs were removed, weighed, immediately frozen in liquid nitrogen and stored at −80° C. until the MPO activity assay was performed. Myeloperoxidase activity was measured using an assay previously described (Krawisz et al., 1984) and modified to be used in a 96-well microtitre plate. Values are expressed as units of MPO activity per milligram of lung tissue (U/mg tissue). For determination of lung fluid content, the left lobes were dissected gently dried using a blotting paper, and weighed. The tissues were dried at 55° C. for 72 h and reweighed. The percentage of lung weight attributed to fluid was calculated as an indicator of lung edema formation.

### (C) Measurement of Cytokine Levels.

Anaesthetized mice from Example 1 were washed with 70% ethanol under sterile conditions. Blood was collected by cardiac puncture and allowed to clot at room temperature for at least 30 mins. The sample was then centrifuged at 400×g for 10 mins for the retrieval of serum. The serum samples were analyzed for inflammation-relevant cytokines and chemokines by using a Luminex 200 apparatus (Applied Cytometry Systems, UK) and a multiplex mouse cytokine/chemokine kit from Millipore (Massachusetts, USA) according to manufacturer's instructions. The panel included IFNγ, IL-4, IL-5, IL-6, IL-9, IL-10, IL-12p40, IL-12p70, IL-13 and TNFα. The data were analyzed with the StarStation V.2.3 software from Applied Cytometry Systems (UK).

### (D) Results.

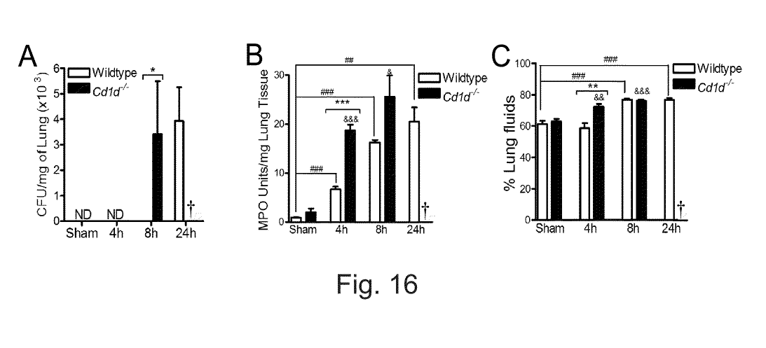

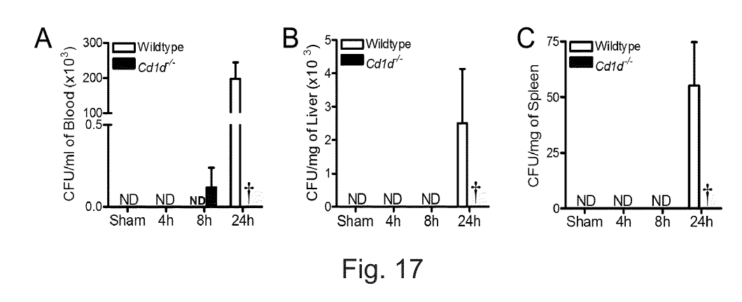

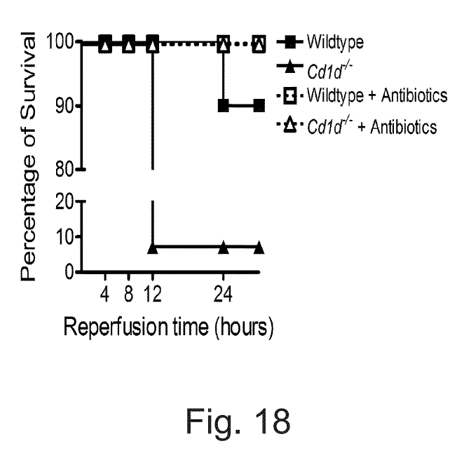

Consistent with current medical views that stroke events trigger an immunomodulatory response that decreases the antimicrobial drive of the immune system in humans, all wild-type mice developed infections 24 h after MCAO, that were detectable in blood, lung, liver, and spleen tissues (FIG. 17A; FIG. 17). These mice also displayed a significant increase in neutrophilic infiltration into lungs, as measured by myeloperoxidase (MPO) levels, and pulmonary edema (FIGS. 16B, 16C), both hallmark features of pneumonia, the most common infection in humans after stroke. Wild-type mice always demonstrated some mortality after MCAO throughout the study (FIG. 18), nearly identical to human mortality data of 12% to 14% (Lichtman et al., 2011, Outcomes after ischemic stroke for hospitals with and without Joint Commission-certified primary stroke centers. Neurology 76:1976-1982; Roger et al. 2011, Executive Summary: Heart Disease and Stroke Statistics—2011 Update: A Report From the American Heart Association. Circulation 123:459-463).

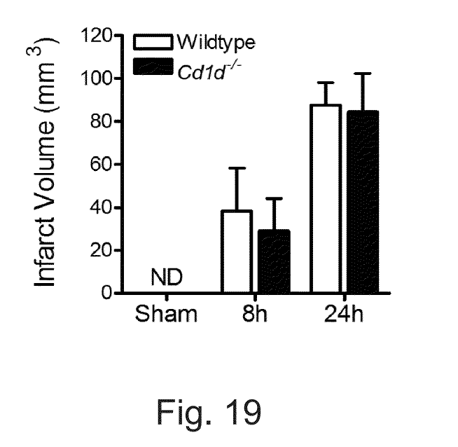

To investigate the role of iNKTcells in stroke and the associated systemic bacterial infection and tissue injury, mice deficient in iNKT cells (Cd1d^{-/-}) were also subjected to MCAO. Bacterial cultures from blood and lungs were clearly evident as early as 8 h after MCAO in Cd1d^{-/-} mice (FIG. 16A; FIG. 17A). Cd1d^{-/-} mice developed even greater pulmonary damage as early as 4 h after MCAO, with more prominent pulmonary neutrophil infiltration (FIG. 16B) and edema (FIG. 16C), suggestive of even earlier pneumonia-like symptoms. The majority of post-ischemic Cd1d^{-/-} mice did not survive past 12 h of reperfusion (FIG. 18); this occurred despite the fact that both strains of mice showed similar brain infarct size (FIG. 19).

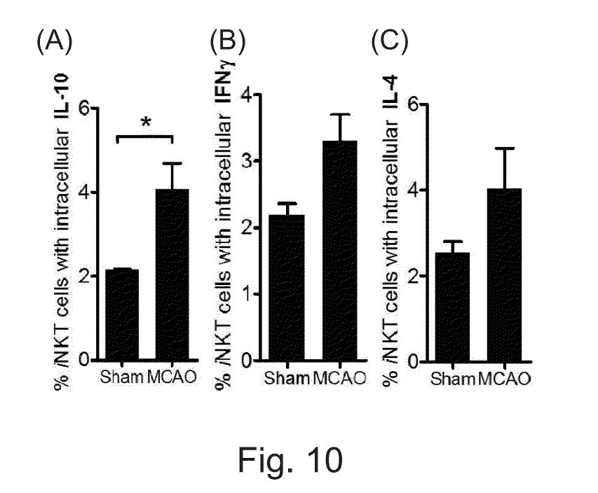

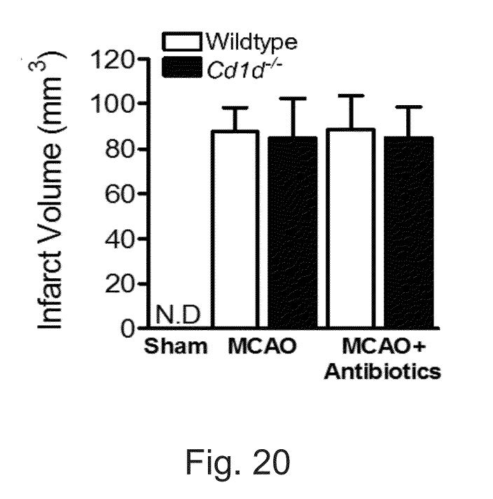

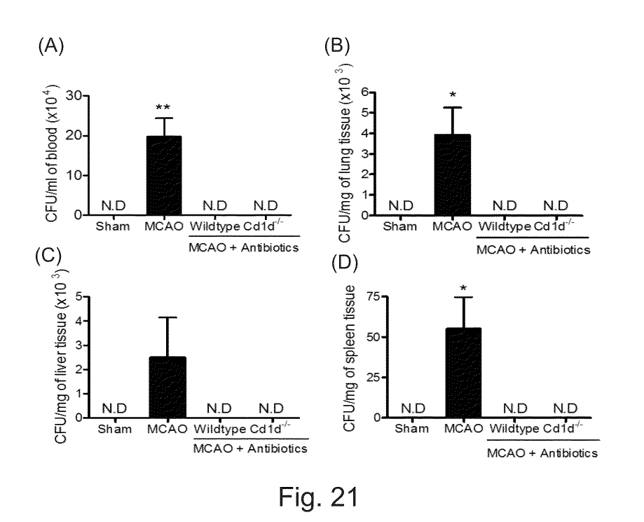

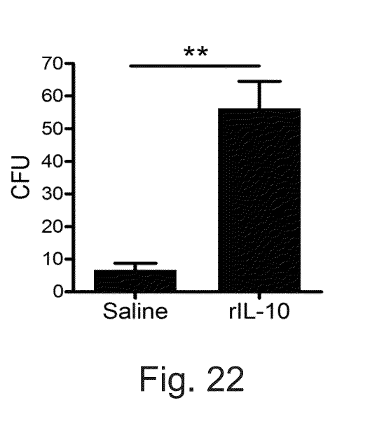

We hypothesized that the high mortality rate of post-ischemic Cd1d^{-/-} mice was the result of their increased susceptibility to post-stroke infections. Indeed, prophylactic administration of antibiotics in post-ischemic mice dramatically improved the survival rate of both strains of mice (FIG. 18). Most striking was the increase in survival of post-ischemic Cd1d^{-/-} mice. The antibiotic treatment did not affect the infarct size of the brain lesion after MCAO but completely prevented the infections in post-ischemic wild-type and Cd1d^{-/-} mice (FIGS. 20, 21). Moreover, wild-type mice pretreated with recombinant IL-10, a TH2-type cytokine that iNKT cells were shown to produce after MCAO (FIG. 10), developed increased stroke-induced lung infections (FIG. 22). Clearly, stroke-activated iNKT cells continued to function and afforded some protection to the host, whereas a complete absence of iNKT cells rendered the animals even more susceptible to post-stroke infections, consistent with the lower T_{H}1-type cytokine levels observed in these mice, including no detectable IFN-γ (FIGS. 11, 15).

### Example 5

### Assessments of Relationships Between iNKT Cells and Peripheral Lymphocyte Changes in Post-Stroke Mice

### (A) Flow Cytometric Analysis.

Blood was collected from anaesthetized mice by cardiac puncture, red blood cells were lysed with ACK lysis buffer (Lonza, Switzerland) and leukocytes were washed in cold FACS wash buffer (FWB; PBS, 2% fetal calf serum, 0.5 mM EDTA). Mice were euthanized and organs were collected into cold FWB. Single cell suspensions were generated from liver, spleen, thymus, and peripheral lymph nodes by mechanical disruption through a 40-μm nylon mesh. Liver cell suspensions were washed with cold PBS, resuspended in 44% isotonic Percoll (Sigma) and layered over 70% isotonic Percoll. Gradients were resolved through centrifugation for 20 min at 400×g in a bench-top centrifuge with no brake, after which the interface containing the lymphocytes was collected and washed in FWB.

For enumeration of leukocyte populations, single cell suspensions of peripheral blood, liver, spleen, thymus and lymph nodes from sham-operated and post-ischemic wild-type mice and Cd1d−/− mice mice were stained with PerCP-conjugated anti-CD45 (30-F11, BD Pharmingen), Alexa Fluor 450-conjugated anti-CD3 (145-2C11, eBioscience), PE-conjugated anti-B220 (RA3-6B2, BD Pharmingen), PE-conjugated anti-CD4 (RM4-4, eBioscience), PE-conjugated anti-NKp46 (29A1.4, eBioscience), PE-conjugated PB57-loaded CDId-tetramer (NIH, Atlanta, Ga.) and eFluor 605NC-conjugated anti-CD8 (53-6.7, eBioscience).

All samples were analysed on an Attune acoustic focusing cytometer (Applied Biosystems).

### (B) Measurement of Blood Flow Velocity.

Blood flow velocity within post-sinusoidal venules was determined via analysis of the velocity of 1-μm diameter inert fluorescent microspheres (yellow/green, PolyFluor; Polysciences, Inc., Warrington, Pa.) injected intravenously, as previously described (Lister and Hickey, 2006). Beads were visualized on spinning-disk confocal microscopy and video sequences of microspheres in the bloodstream were recorded and analyzed. Perfusion velocity was determined by quantification of the velocity of 30 individual beads per vessel, three vessels per mouse.

### (C) Localized Noradrenalin Superfusion.

To demonstrate that noradrenergic stimulation directly affects the behavior of iNKT cells, induced a localized noradrenalin superfusion was induced on the liver of Cxcr6^{gfp/+} mice. Alexa Fluor 647-conjugated rat anti-mouse PECAM-1 (clone 390; 1 μl) purchased from eBioscience (San Diego, Calif., USA) was mixed with the noradrenalin (5 μg; Sigma) to enable visualization of the epicentre of noradrenalin superfusion. Using intravital spinning-disk confocal microscopy, the behavior of iNKT cells was visualized and tracked in the epicentre of, and distant from the localized noradrenalin in the livers of Cxcr6^{gfp/+} mice.

(D) In Vitro Isolation of iNKT Cells.

Liver-derived lymphocytes and spleen-derived lymphocytes were isolated from Cxcr6^{gfp/+} mice following the method taught by Ajuebor et al. (2003, C-C Chemokine Ligand 2/Monocyte Chemoattractant Protein-I Directly Inhibits NKT Cell IL-4 Production and Is Hepatoprotective in T Cell-Mediated Hepatitis in the Mouse. J. Immunol. 170(10):5252-5259). Briefly, livers and spleens were excised and finely minced in a digestive medium containing 0.05% collagenase type IV (Worthington Biomedical Corp.) and 0.002% DNase I in HBSS. After gentle agitation at 37° C. for 30 min, the concentrate was passed through a 40-μm nylon filter and washed twice with ice-cold PBS (pH 7.4) and centrifuged at 400×g for 10 min. Lymphocytes were purified on a 44%/70% Percoll gradient and were negatively selected with anti-Gr-1 (RB6-8C5; eBioscience), anti-F4/80 (BM8; eBioscience), anti-CD19 (1D3; eBioscience), and anti-CD8a (53-6.7; eBioscience) microbeads and MiniMACS column (Miltenyi Biotech) to remove neutrophils, macrophages, B cells, and CD8+ cells, respectively. A pure iNKT cell population consisting of TCRf3+ and GFP+ cells were sorted. Purity of isolated iNKT cells was >95%.

iNKT cells were seeded at 5×105 cells into plasma-coated 6 cm clear flat glass bottom dishes and allowed to settle in DMEM containing 10% FCS for 1 h. To examine noradrenalin activation of iNKT cells in vitro, iNKT cells were either pretreated with 400 μM propranolol and then treated with 200 μM noradrenalin or the iNKT cells were subjected to 200 μM noradrenalin alone. The behavior of iNKT cells was visualized and recorded with FluoView FV1000 confocal fluorescence microscope (Olympus). The recorded images were randomized and analyzed by three blinded investigators.

### (E) Results.

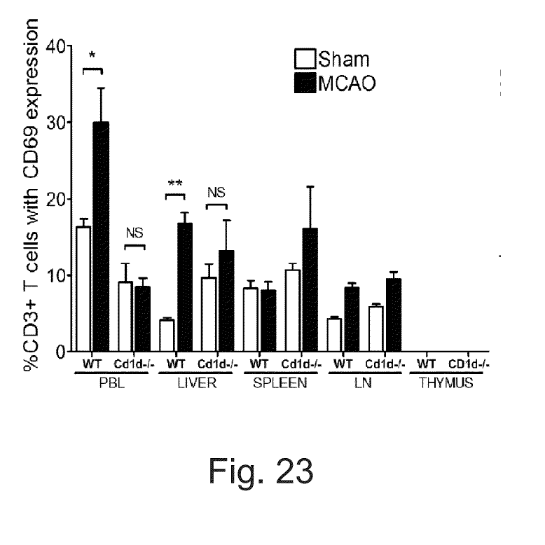

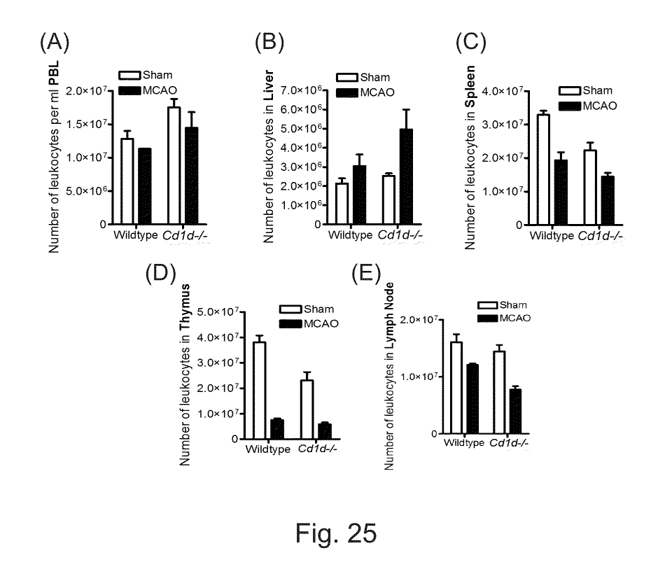

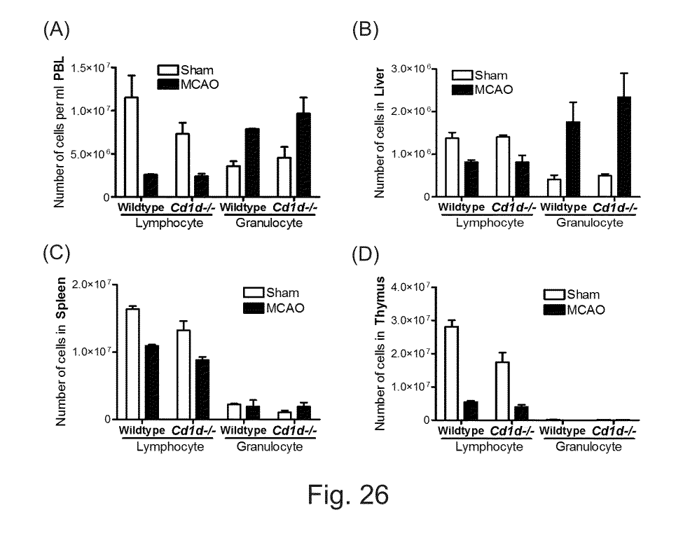

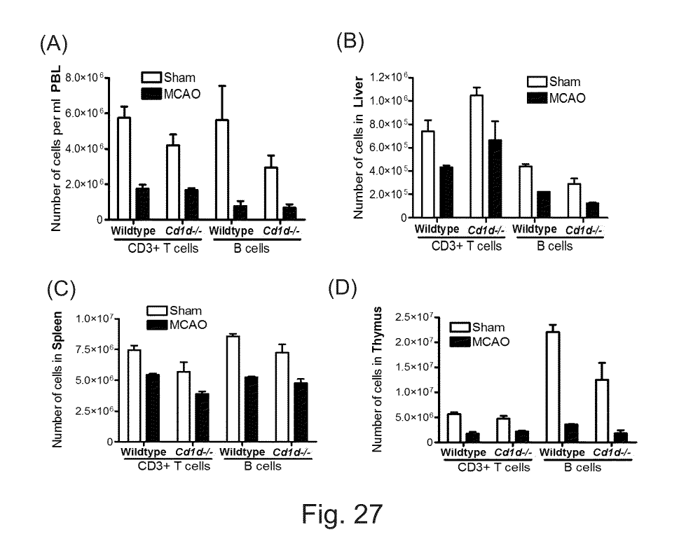

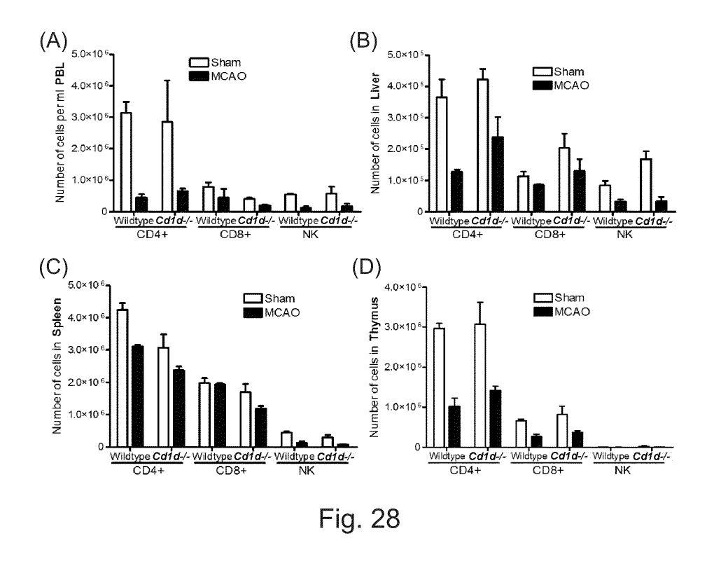

Assessments were done to determine where iNKT cells fit into the previously described peripheral lymphocyte changes in post-stroke mice, following the procedures taught by Prass et al. (2003, Stroke-induced immunodeficiency promotes spontaneous bacterial infections and is mediated by sympathetic activation reversal by poststroke T helper cell type 1-like immunostimulation. J. Exp. Med. 198:725-736) and Offner et al. (2006, Experimental stroke induces massive, rapid activation of the peripheral immune system. J. Cereb. Blood. Flow Metab. 26:654-655). T cell activation (CD69 expression) was increased in the peripheral blood and liver after stroke in wild-type but not Cd1d^{-/-} mice (FIG. 23). In fact, Cd1d^{-/-} mice failed to activate CD4+ T cells in the peripheral blood (FIG. 24A) and CD8+ T cells in the liver after MCAO (FIG. 24B), tissues where iNKT cells were observed to be activated after stroke (FIG. 9). These data suggest that the immune regulation of post-ischemic iNKT cells is upstream of CD4+ and CD8+ T cell activation in the peripheral blood and liver, respectively, and that iNKT cells function as the conductor of immunity, whereby their acute responses modulate and facilitate the adaptive immune response. Although a systematic assessment of numbers of lymphocytes, NK cells, and granulocytes after stroke revealed some additional changes in blood and tissues, these were not affected by the presence or absence of iNKT cells (FIGS. 25, 26, 27, 28), suggesting that not all changes to leukocyte cell numbers are modulated by iNKT cells.

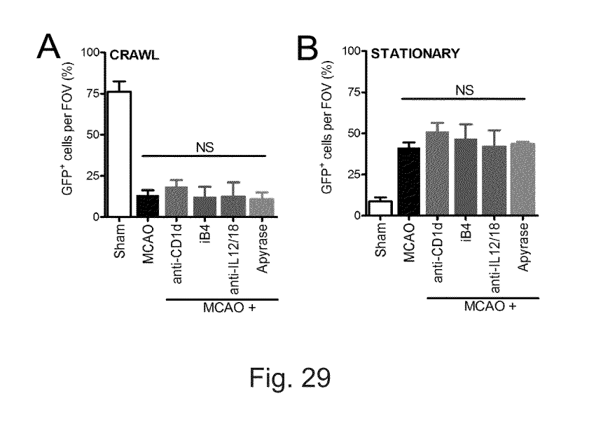

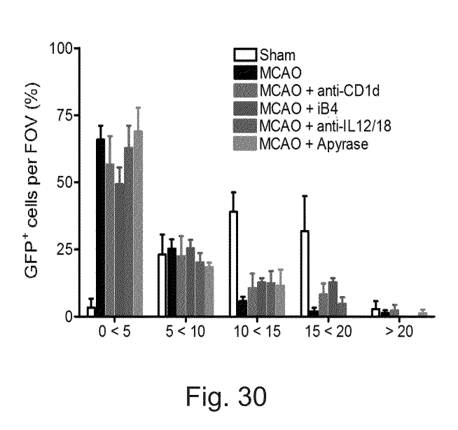

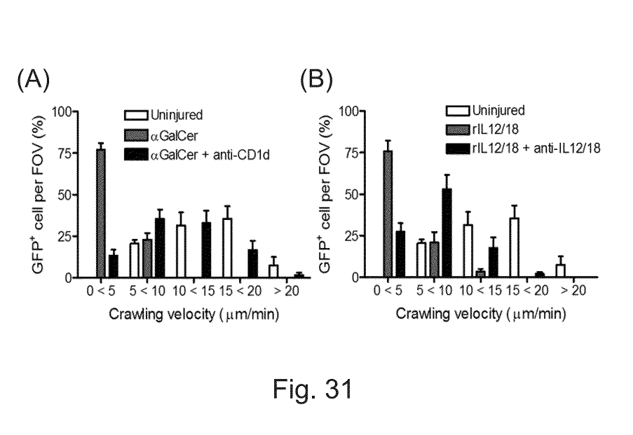

The manner in which iNKTcells detect tissue damage after stroke could be through endogenous glycolipids presented by CD1d, cytokines like IL-12 and/or IL-18 released from other sentinel cells (e.g., macrophages), or some other as-yet-unknown mechanism. Antibody blockade of CD1d had no effect on iNKT cell arrest in response to MCAO (FIGS. 29, 30), whereas it prevented cessation of iNKTcells caused by stimulation with the CD1d ligand α-GalCer, a specific activator of iNKT cells (FIG. 31A). Another inhibitor that blocks the presentation of glycolipid ligands in the context of CD1d, isolectin B (iB) 4, also did not alter iNKT cell arrest after MCAO (FIGS. 29, 30), ruling out glycolipid presentation by CD1d as the pathway alerting iNKT cells to distal tissue injury in stroke. Furthermore, blockade of IL-12 and IL-18, cytokines known to activate and arrest iNKTcells (FIG. 31B), had no effect on iNKT cell arrest in response toMCAO (FIGS. 29, 30). Finally, apyrase, an inhibitor of ATP, a well-known “alarmin” in the brain and liver, had no effect on iNKTcell responses to MCAO (FIGS. 29, 30).

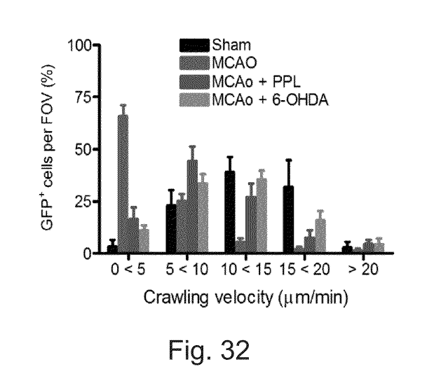

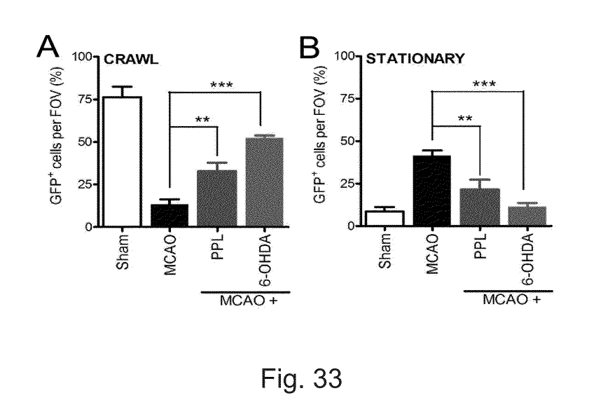

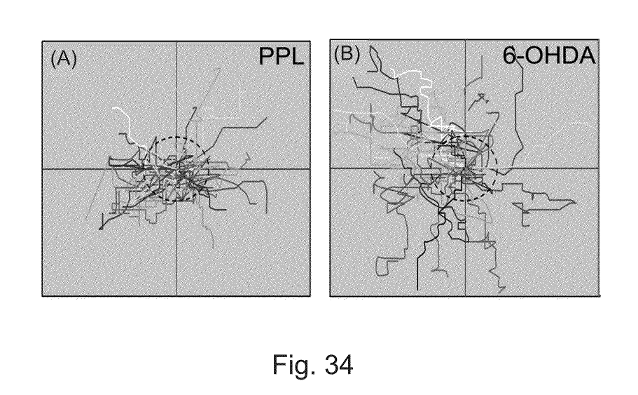

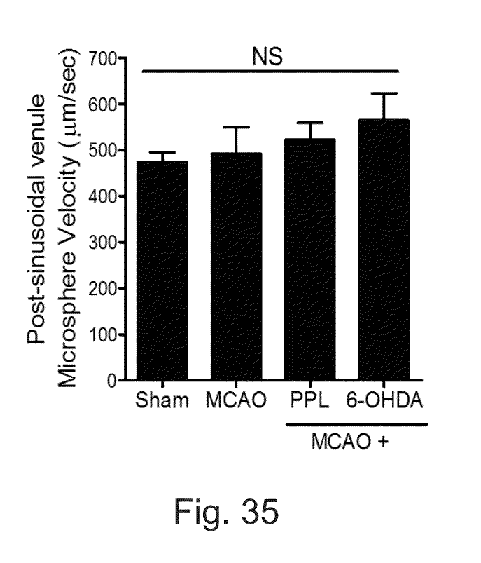

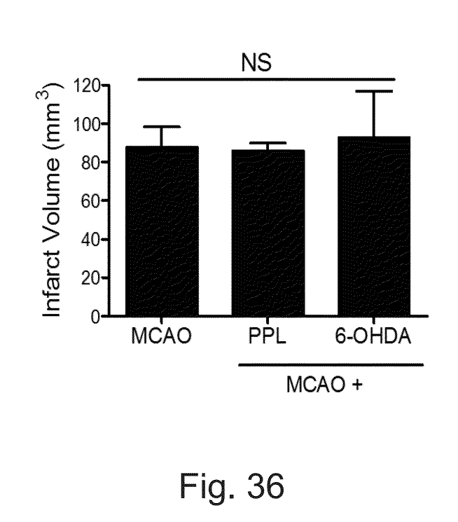

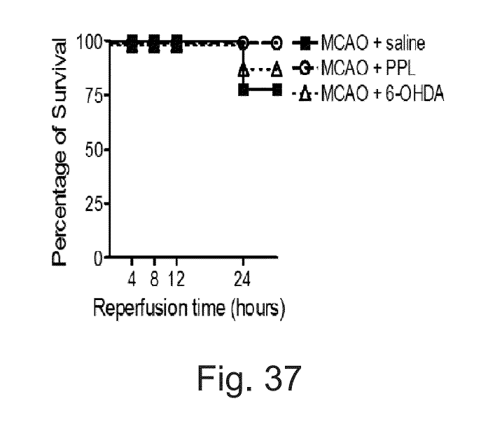

An as-yet-unidentified, long-distance pathway was affecting the crawling behavior and activation of iNKT cells in the liver after cerebral ischemia. Previous literature suggested that the nervous system may affect immune cells, including iNKT cells, and alter their function (Minagawa et al.; 2000, Intensive expansion of natural killer T cells in the early phase of hepatocyte regeneration after partial hepatectomy in mice and its association with sympathetic nerve activation. Hepatology 31(4):907-915; Oya et al., 2000, The differential effect of stress on natural killer T (NKT) and NK cell function. Clin. Exp. Immunol., 121(2):384-390), thereby potentially regulating the magnitude of the host response to infection or injury (Steinman, 2004, Elaborate interactions between the immune and nervous systems. Nat. Immunol., 5(6):575-581; Tracey, 2009, Reflex control of immunity. Nat. Rev. Immunol., 9(6):418-428). Administration of the nonspecific β-adrenergic receptor blocker, propranolol, reversed the iNKT cell phenotype induced by MCAO (FIGS. 32, 33, 34A). Furthermore, post-ischemic cessation of iNKT cell crawling was completely inhibited by specific chemical depletion of peripheral neuronal terminals containing noradrenalinewith 6-hydroxydopamine (6-OHDA), suggesting a neural rather than humoral input (FIGS. 32, 33, 34B). Despite the phenotypic changes of iNKT cells after systemic administration of propranolol or 6-OHDA, these treatments did not alter the blood flow (FIG. 35) or infarct size in post-ischemic mice (FIG. 36). Mortality at 24 h of reperfusion was reduced by 50% with 6-OHDA and completely inhibited by propranolol (FIG. 37).

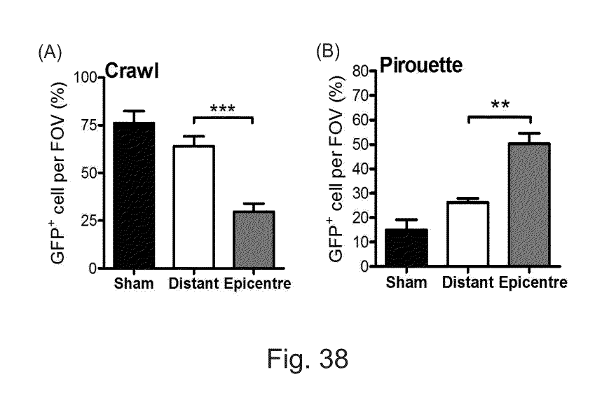

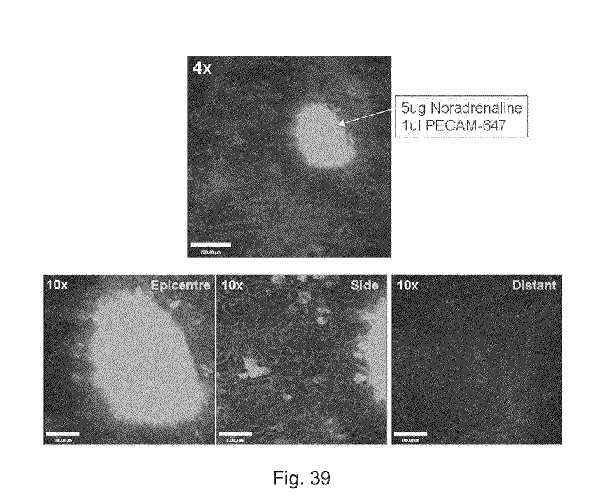

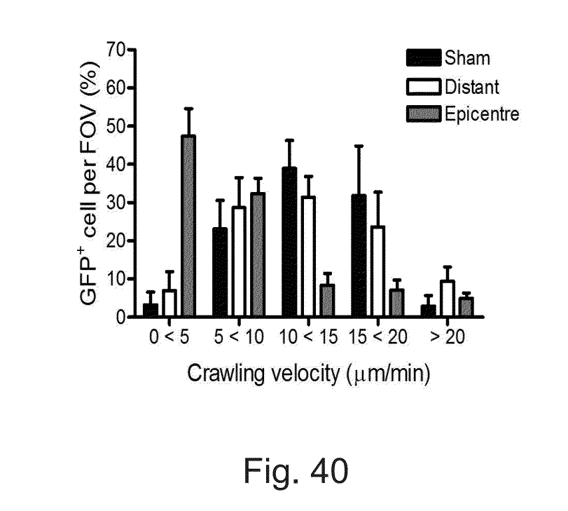

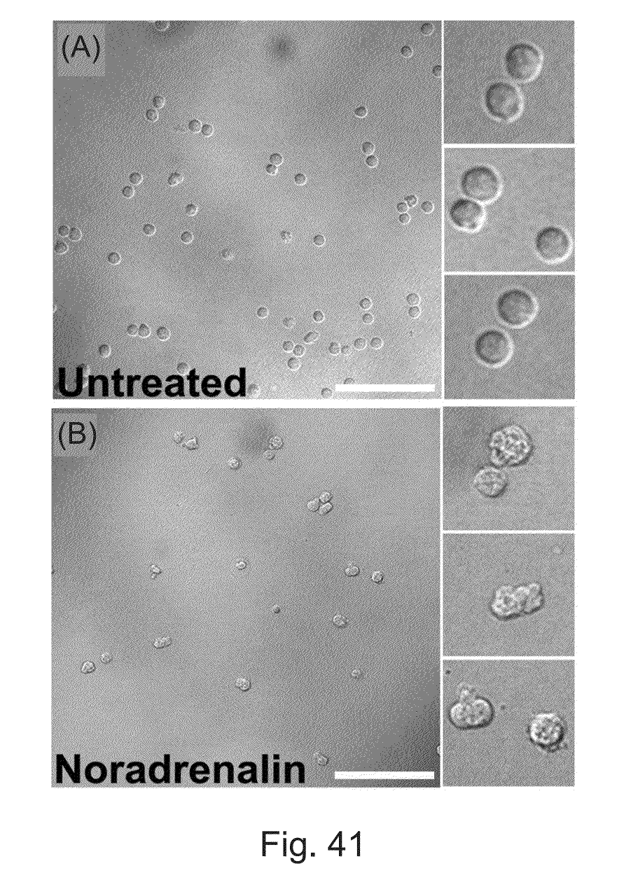

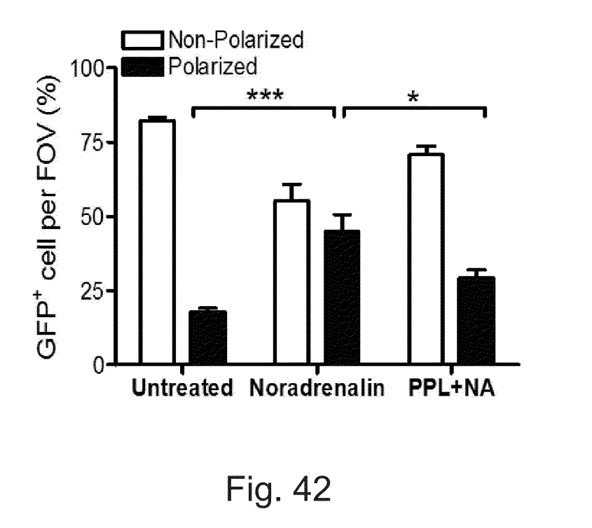

Localized noradrenaline administration directly mimicked the behavior of iNKT cells in the liver of post-ischemic Cxcr6^{gfp/+} mice in vivo. Significantly fewer iNKT cells crawled, and more cells adopted a pirouetting phenotype in the epicenter of noradrenaline administration (FIGS. 38, 39, 40). By contrast, in an area of liver distant from the localized noradrenaline superfusion, iNKT cells did not alter their crawling behavior (FIGS. 38, 39, 40). In addition, when noradrenaline was applied to iNKT cells in vitro, these cells acquired a “flattened” profile and developed a pseudopod-protruding phenotype reminiscent of iNKT cell behavior after MCAO in vivo (FIG. 41). In fact, pretreatment of iNKT cells with propranolol inhibited this behavioral change (FIG. 42), suggesting that noradrenaline directly induces the biology observed in vivo.

### Example 6

### Assessments of Effects of iNKT Cells on Stroke-Induced Immunosuppression and Infection in Post-Stroke Mice

### (A) Experimental.

Cxcr6^{gfp/+} mice were separated into three groups three days prior to administration of the MCAO procedure. The first group of mice were injected with 6-hydroxydopamine (6-OHDA; Sigma) to chemically sympathectomize peripheral nerve terminals containing noradrenalin. The 6-OHDA was injected intraperitoneally at a concentration of 250 mg/kg 3 days prior to MCAO surgery. The remaining mice underwent the MCAO procedure as previously described in Example 1. Two hours after the MCAO procedures, the post-ischemic Cxcr6^{gfp/+} mice were separated into two groups referred to in this section as the “second group” and the “third group”. Mice from the second group were each injected with the α-GalCer. Mice from the third group were each injected with propanolol. The propanolol was dissolved in 0.9% sodium chloride at 3 mg/ml, and administered at 30 mg/kg body weight at 4 and 8 h after MCAO. Control sham-operated mice were treated as previously described in Example 1. Each treatment group in this study consisted of 4 mice.

### (B) Assessments of Stroke-Induced Lung Injuries.

Twenty four hours after the MCAO procedures, all mice were anaesthetized, then washed with 70% ethanol under sterile conditions.

Levels of the cytokine IFNγ present in peripheral blood were determined by collecting blood samples by cardiac puncture, which were then allowed to clot at room temperature for at least 30 mins. The samples were then centrifuged at 400×g for 10 mins for the retrieval of serum. The serum samples were analyzed for the cytokine IFNγ by using a multiplex mouse cytokine/chemokine kit, and a Luminex 200 apparatus according to manufacturer's instructions. The data were analyzed with the StarStation V.2.3 software from Applied Cytometry Systems (UK).

The lung tissues were removed at 24 h post-MCAO and assessed for: (i) myeloperoxidase activity as a measure of neutrophil infiltration, and (ii) for lung edema.

### (C) Bacteriological Analysis.

The anaesthetized mice from Example 1 were washed with 70% ethanol under sterile conditions. Blood was collected by cardiac puncture. The lungs, liver and spleen were removed after thoracotomy and homogenized. For determination of colony forming units (CFU), 10 μl of tissue homogenate or blood was serially diluted, plated onto brain heart infusion (BHI) agar plates supplemented with 5% sheep blood (Daylnn Biologicals, Alberta, Canada), incubated at 37° C. for 18 h, and bacterial colonies were counted.

### (D) Results.

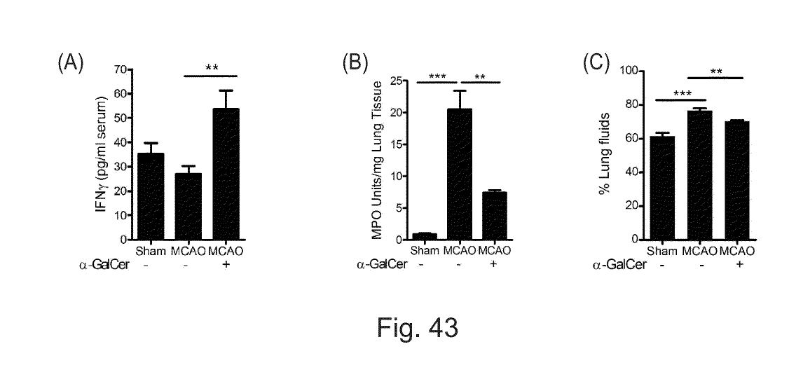

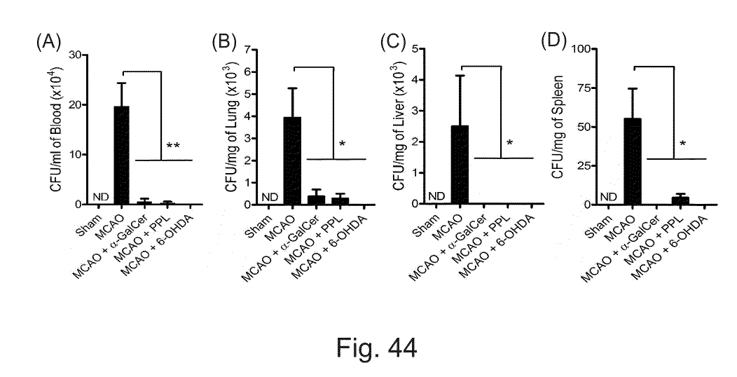

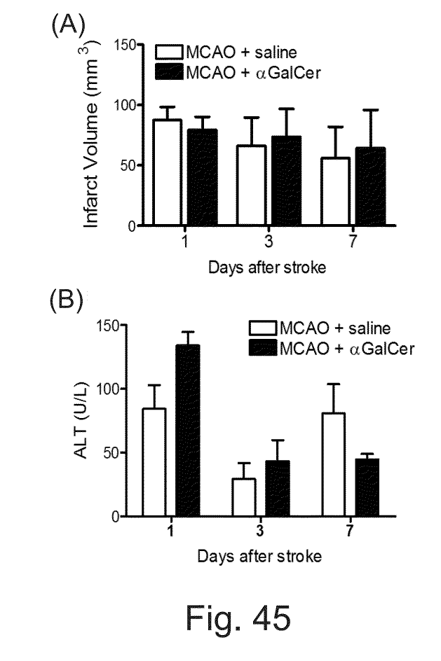

Administration of α-GalCer in a therapeutically relevant manner significantly increased systemic levels of endogenous IFN-γ (FIG. 43A) and reduced stroke-induced neutrophil pulmonary influx, lung edema (FIGS. 43B, 43C), and infections in post-ischemic mice (FIG. 44A-44D). α-GalCer is an immunostimulant that could potentially have deleterious effects on cerebral ischemia, but the inventors found no notable differences in infarct sizes (FIG. 45A). Furthermore, α-GalCer has been documented to induce liver damage, but the inventors found no significant difference in liver enzyme levels within the blood of post-ischemic mice after the single dose of α-GalCer (FIG. 45B).

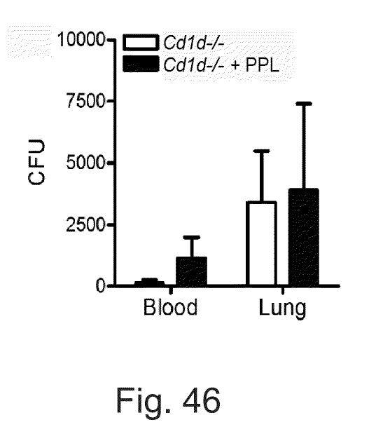

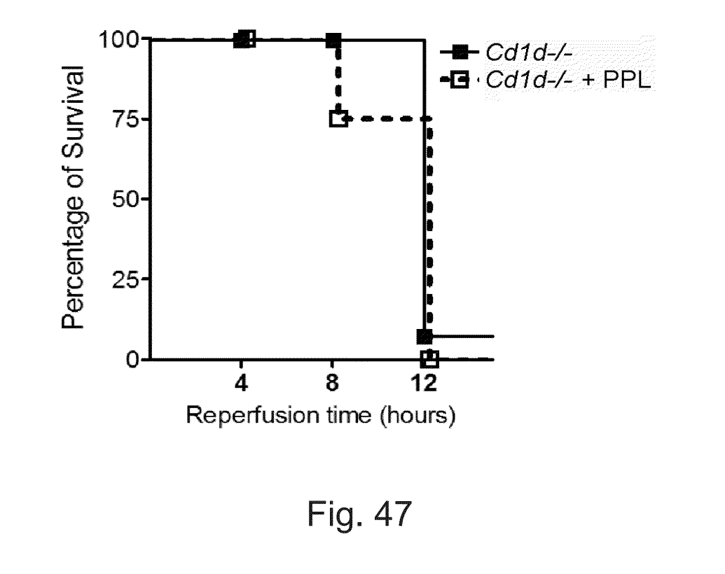

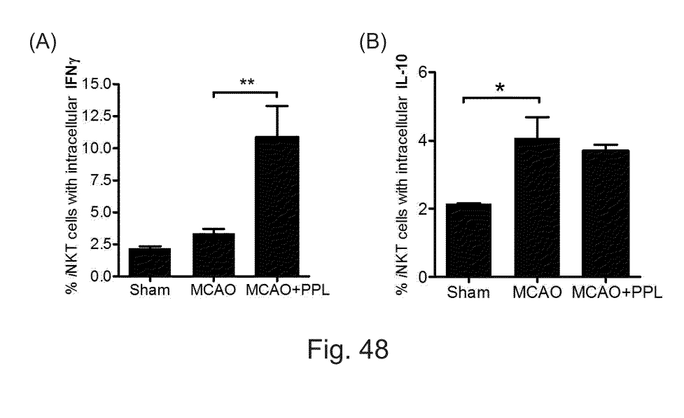

Interestingly, wild-type mice receiving propranolol or 6-OHDA also demonstrated significantly reduced bacterial infections at 24 h after MCAO in a manner similar to that observed in α-GalCer-treated mice (FIGS. 44A-44D). Furthermore, the effects of propranolol were entirely dependent on iNKT cells, because the addition of propranolol to Cd1d^{-/-} mice provided no protection from infection or mortality (FIGS. 46, 47). Notably, post-ischemic wild-type mice treated with propranolol reversed the preference for intracellular IL-10 production back to an intracellular IFN-γ dominant production and toward a T_{H}1-dominant phenotype (FIG. 48). These data strongly suggest that direct modulation of iNKT cells with α-GalCer or through the blockade of noradrenergic neurotransmitters was sufficient to modulate iNKT cells in a manner that results in reduced infection and associated lung injury after stroke.

### Example 7

### Assessments of Effects of α-GalCer Analogs and α-GalCer Derivatives on Crawling Behavior of NKT Cells

### (A) Experimental.

Wildtype mice underwent the MCAO procedure as previously described in Example 1. In separate experiments, each of the α-GalCer compounds listed in Table 1 was dissolved in DMSO to a final concentration of 2 μM. Sham-operated control mice and post-ischemic mice were injected with 2 μg of a selected composition at about 2 h after performance of the MCAO procedures (4 mice/treatment). The control group was sham-operated control mice injected with volumes of DMSO equal to the volumes used for the experimental treatments. Additionally, separate groups of post-ischemic mice were injected with 4 μg of an α-GalCer composition comprising SK08-25 or SK08-27 or SK08-29 or SK08-30, about 2 h after performance of the MCAO procedures. The crawling behaviours of crawling GFP+ cells in livers from sham-operated control mice and post-ischemic mice were observed and quantified using the methods disclosed in Example 2.

### (B) Results.

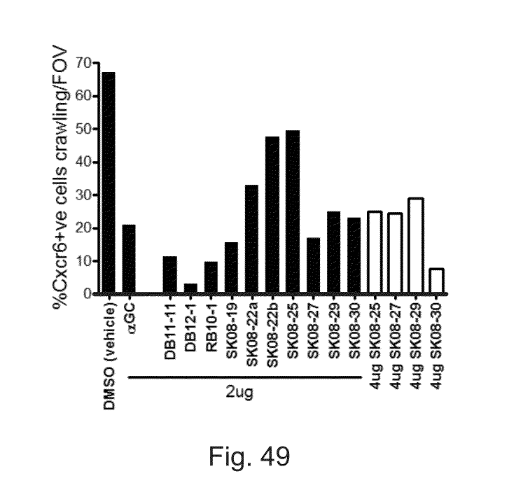

The data in FIG. 49 indicate that all 11 α-GalCer compositions administered at 2 μg and the four α-GalCer compositions administered at 4 μg reduced the crawling behaviour of NKT cells in livers compared to the crawling behaviour of NKT cells in livers treated only with the DMSO vehicle. These data may be interpreted to infer that the reduction of α-GalCer-induced crawling behaviour in livers of mice is the consequence of the T cell receptor (TCR) molecules on the surfaces of NKT cells being engaged by antigen-presenting CD1 glycoproteins of anaphase-promoting complexes (APC). The result is activation of the NKT cells for rapid production and release of immunomodulary cytokines such as exemplified by IL-2, IFN-γ, TNF-α, and IL-4, that directly affect and promote various immune responses.

### Example 8

### Assessments of Effects of α-GalCer, α-GalCer Analogs and α-GalCer Derivatives on Occurrences of Microbial Infections

### (A) Experimental.

The anaesthetized mice from Example 7 were washed with 70% ethanol under sterile conditions. Blood was collected by cardiac puncture. The lungs, liver and spleen were removed after thoracotomy and homogenized. For determination of colony forming units (CFU), 10 μl of tissue homogenate or blood was serially diluted, plated onto brain heart infusion (BHI) agar plates supplemented with 5% sheep blood (Daylnn Biologicals, Alberta, Canada), incubated at 37° C. for 18 h, and bacterial colonies were counted.

### (B) Results.

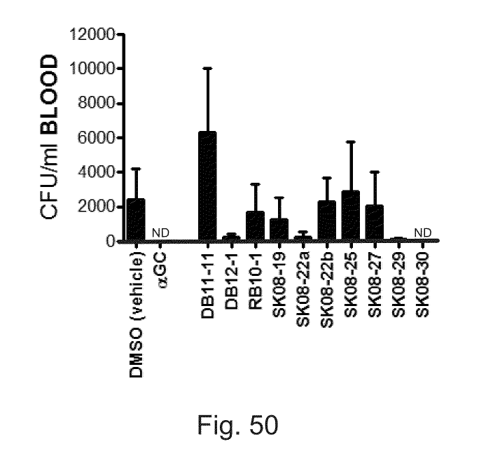

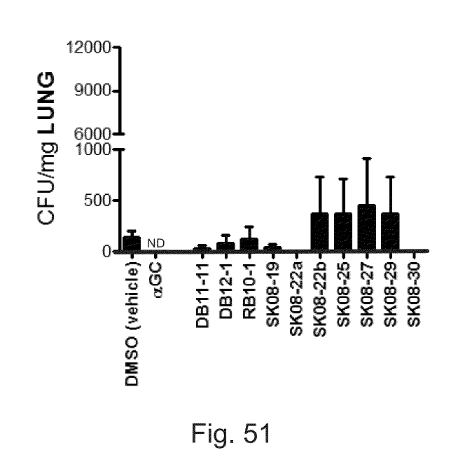

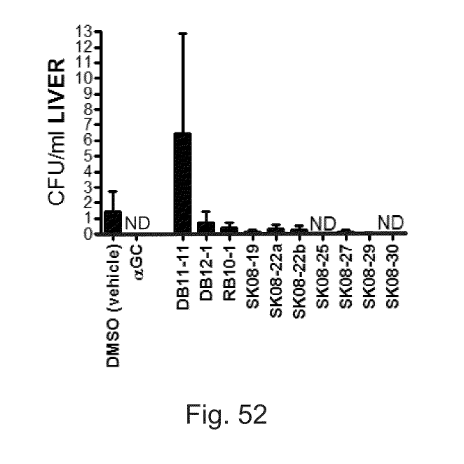

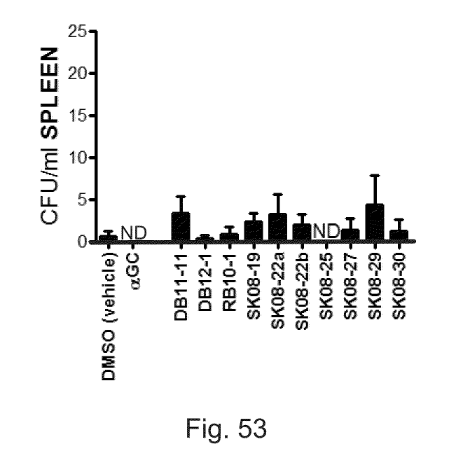

The data in FIGS. 50-53 show that administration of a single dose of all 11 α-GalCer compositions to post-ischemic mice about 2 h after the stroke events reversed the susceptability of these mice to infection. The data in FIG. 50 show that the bacterial loads in peripheral blood samples collected from post-ischemic mice receiving dosages of the different α-GalCer compositions were not significantly different from the bacterial loads in the control samples. The data in FIG. 51 show that the bacterial loads in lungs collected from post-ischemic mice receiving dosages of the different α-GalCer compositions were not significantly different from the bacterial loads in the control samples. The data in FIG. 52 show that the bacterial loads in livers collected from post-ischemic mice receiving dosages of the different α-GalCer compositions were not significantly different from the bacterial loads in the control samples. The data in FIG. 53 show that the bacterial loads in spleens collected from post-ischemic mice receiving dosages of the different α-GalCer compositions were not significantly different from the bacterial loads in the control samples.

### Example 9

### Assessments of α-GalCer, α-GalCer Analogs and α-GalCer Derivatives on Alanine Transaminase Activity in Controls and in Post-Ischemic Subjects

### (A) Experimental.

Separate groups of control sham-operated mice received a single-dose administration of the α-GalCer compositions following the procedures, both as outlined in Example 7 (one composition/one group of four mice). Also, separate groups of post-ischemic mice received a single-dose administration of the α-GalCer compositions about 2 h after the MCAO events, following the procedures as outlined in Example 7 (one composition/one group of four mice). All animals were euthenized 24 h after MCAO events, and their peripheral blood was harvested. The levels of alanine transaminase (ALT) in the samples were determined with an ALT activity assay following the manufacturer's instructions to determine the rates of oxidation of NADH to NAD⁺.

### (B) Results.

ALT is a homodimeric cytoplasmic pyridoxal phosphate-dependent enzyme involved in cellular nitrogen metabolism, amino acid metabolism, and liver gluconeogenesis. ALT mediates conversion of major intermediate metabolites, catalyzing reversible transamination between alanine and α-ketoglutarate to form pyruvate and glutamate. ALT is widely distributed in many tissues but is found in greatest abundance in the liver, and to a much lesser extent in the kidneys, heart, and brain. The major role of ALT in the liver is the conversion of alanine to glucose which is then exported to the body to be utilized in a multitude of processes. ALT levels are generally low, but may spike during disease states or in the event of tissue injury. As such, ALT levels are routinely used as indicators of medical issues, particularly liver diseases.

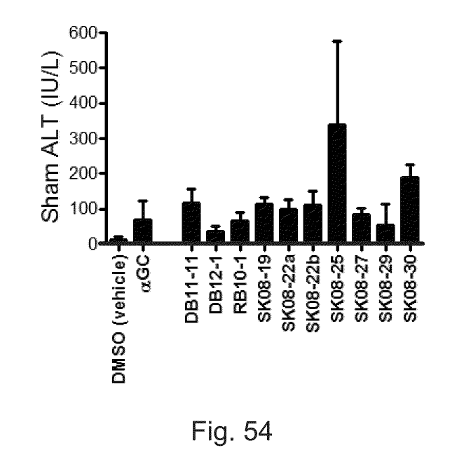

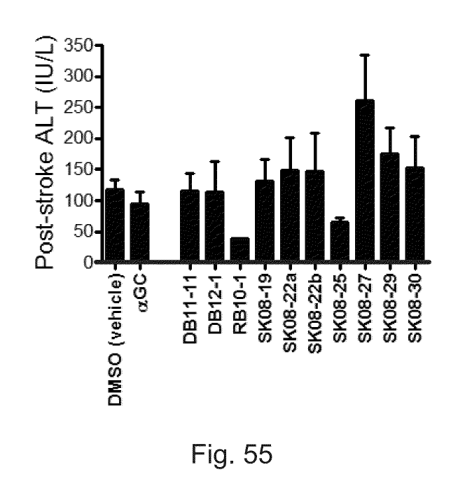

The data in FIG. 54 indicate that the individual α-GalCer compositions appeared to cause some increases in the rates of NADH oxidation in the control sham-operated mice. However, the data in FIG. 55 indicate that: (i) the rates of NADH oxidation increased in post-ischemic mice receiving the control DMSO dosage, and (ii) none of the α-GalCer compositions administered about 2 h after MCAO had any significant effects on ALT activity in reference to the DMSO control data.

### Example 10

### Assessments of the Effects of Stroke Events on (i) NKT Cell Activation in Peripheral Blood, and (ii) on Plasma Cytokine Levels in Human Subjects

### (A) Experimental.

In human stroke, the balance between pro-inflammatory and anti-inflammatory cytokines is an important prognostic clinical factor. In mouse models, iNKT cells that reside within blood vessels and function as master regulators of immune response, The behavior and function of iNKT cells change in the behavior and function following a cerebral ischemia-reperfusion injury in mice. Since iNKT cells function to bridge innate and adaptive immunity and act as master regulators of immunity, the following assessments were made to determine if the roles of iNKT cells in human subjects are also altered as a consequence of stroke-mediated immunosuppression.

Accordingly, this study compared peripheral blood profiles of human stroke victims with: (i) a hospital control patient group that had not experienced a brain trauma prior to the start of the study, and (ii) a non-hospitalized healthy group. A group of 37 stroke patients were recruited and initial peripheral blood samples were collected within 24 h of their stroke event. Additionally, a group of non-stroke hospitalized patients and a group of non-hospitalized healthy subjects were recruited, and peripheral blood samples were collected from each individual. The initial blood samples were designed as “Time 0” (also denoted as “baseline”). Blood samples were subsequently collected from each individual at 1 day, 2 days, 7 days (or on discharge), and 90 days after Time 0. The following assessments, using the procedures disclosed in Example 3 for blood flow cytometry analysis, were done with each blood sample collected: (i) determination of the iNKT cell activation status, (ii) determination of the systemic cytokine profiles, and (iii) determine if any correlations existed between iNKT activation, cytokine profiles, infection rates, and (iv) if the patient was on adrenergic blockers.

### (B) Results.

An important aspect of flow cytometry data analysis is the use of gating procedures to selectively visualize the cells of interest while eliminating results from unwanted particles e.g. dead cells and debris. Cells have traditionally been gated according to physical characteristics. For instance, subcellular debris and clumps can be distinguished from single cells by size, estimated by forward scatter. Also, dead cells have lower forward scatter (FSC) and higher side scatter (SSC) than living cells. The different physical properties of granulocytes, monocytes and lymphocytes allow them to be distinguished from each other and from cellular contaminants.

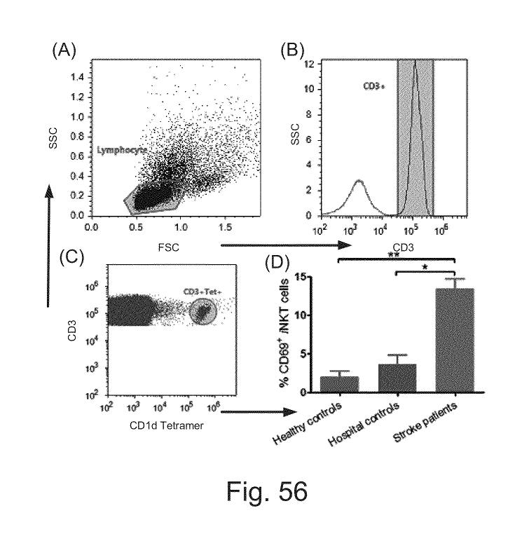

The data in FIG. 56(A) show a SSC scatter plot used for identification of lymphocytes in the human blood samples collected for this study. The data in FIG. 56(B) showsthe selection of lymphocytes expressing CD3+, from the population of NKT cells shown in FIG. 56(A). Subsequently, iNKT cells were identified by a CD1d-loaded tetrameter, shown in FIG. 56(C). The data in FIG. 56(D) is a chart showing the levels of iNKT cell activation, i.e., expressing CD69+, in blood samples collected from the non-hospitalized control subjects, from the hospital control patient group, and from the stroke victims.

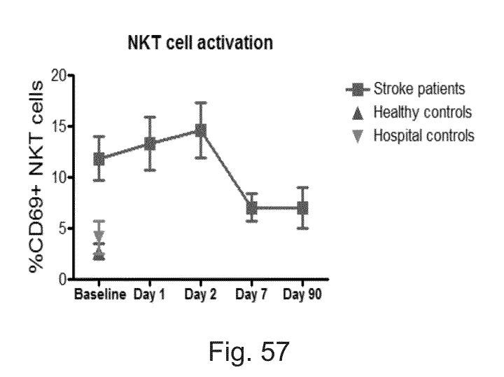

The data in FIG. 57 show that the percentage of NKT cells expressing CD69+ was about 3-fold higher at Time 0 in stroke patients than in the hospital control patient group and the healthy control group. The percentage of NKT cells expressing CD69+ in stroke patients increased during the 48 h after Time 0, and then fell by about 50% 7 days after Time 0. However, the percentage of NKT cells expressing CD69+ in stroke patients was about double the percentages in the hospital control patient group and to the healthy control group. It is noted that the elevated level of NKT cells expressing CD69+ in stroke patients was transient.

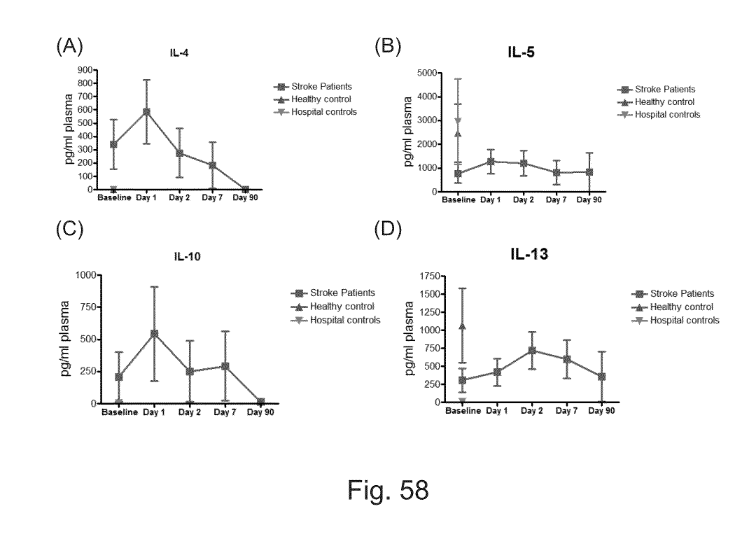

FIG. 58 are charts showing systemic production of T_{H}2 cytokines in stroke patients, hospital control patients, and healthy control subjects wherein (A) shows production of IL-4, (B) shows production of IL-5, (C) shows production of IL-10, and (D) shows production of IL-13. It is noteworthy that the levels of IL-4 (A) and IL-10 (C) increased dramatically during the 7 days after a stroke event, and then returned to approximate levels in the hospital control patients and in the healthy control subjects.

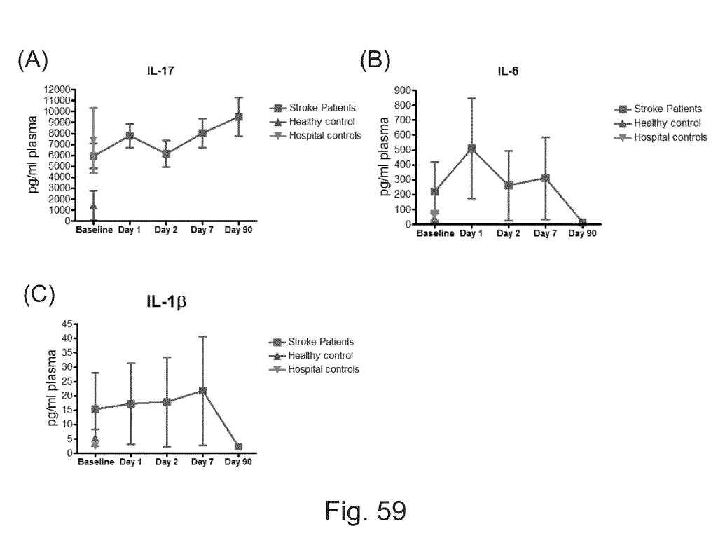

FIGS. 59(A)-59(D) are charts showing systemic production of other cytokines in stroke patients, hospital control patients, and healthy control subjects wherein (A) shows production of IL-17, (B) shows production of IL-6, and (C) shows production of IL-1β. It is noted that the levels of IL-6 (B) and IL-1β (C) increased dramatically during the 7 days after a stroke event, and then returned to approximate levels in the hospital control patients and in the healthy control subjects.

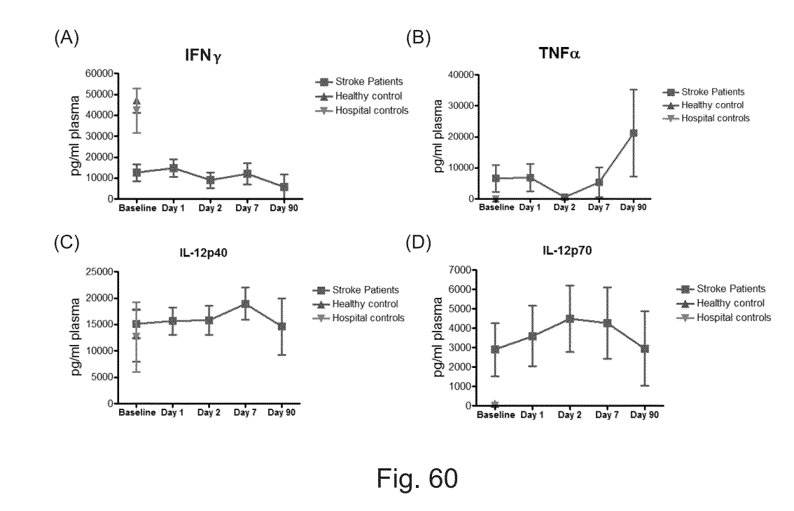

FIG. 60 are charts showing systemic production of T_{H}1 cytokines in stroke patients, hospital control patients, and healthy control subjects wherein (A) shows production of IFNγ, (B) shows production of TNF-α, (C) shows production of IL-12p40, and (D) shows production of IL-12p70. The data in FIGS. 60(B) and 60(D) indicate that production of the cytokines TNF-α and IL-12p70 increased dramatically after stroke events in reference to their production in hospital control patients and healthy control subjects. It appeared that stroke events did not affect the production of IL-12p40 (FIG. 60(C)). Of particular significance, however, the data in FIG. 60(A) indicates that production of IFNγ in stroke victims is reduced by over 75% in the first 24 hrs after the stroke event, in comparison to the production of IFNγ in hospital control patients and in healthy control subjects. Furthermore, production of IFNγ in stroke victims remained significantly diminished over the 90-day monitoring period. We also noted that the human data in FIG. 60(A) is correlated with the observations with mice (FIG. 11(A)) which show significantly decreasing production of IFNγ after MCAO. These data suggest that a functional innervation of iNKT cells in the liver contributes to this immunosuppression. Accordingly, the data in FIG. 43 and in FIG. 44 showing that post-trauma administration of an α-GalCer composition suppressed the occurrence of microbial infections support the view that administration of α-GalCer interferes with and protects against stroke-related innervation of NKT cells in the liver that results in significant reduction in production of IFNγ in stroke victims.

### Example 11

### Assessments of the Effects of Brain Trauma on NKT Cell Activation in Peripheral Blood in Human Subjects

### (A) Experimental.

This study compared peripheral blood profiles in peripheral blood samples collected from human victims suffering one of the following four brain traumas: (i) traumatic brain injury (TBI; 6 subjects); (ii) intracerebral hemorrhage, a type of hemorrhagic stroke (ICH; 1 subject); (iii) subarachnoid hemorrhage, a type of hemorrhagic stroke (SAH; 2 subjects); and (iv) ischemic stroke (“Stroke”; 3 subjects).

Also, blood samples were collected from three control groups: (v) patients from a hospital intensive care unit (ICU; 1 subject); (vi) patients from a hospital cardiovascular intensive care unit (CVICU; 8 subjects); (vii) non-hospitalized healthy group (7 subject). Samples were also collected from an additional group classified as “Seizures” (1 subject).

The following assessments, using the procedures disclosed in Example 3 for blood flow cytometry analysis, were done with each blood sample collected: (i) determination of the iNKT status, (ii) determination of the systemic cytokine profiles, and (iii) determine if any correlations existed between iNKT activation, cytokine profiles and infection rates.

### (B) Results.

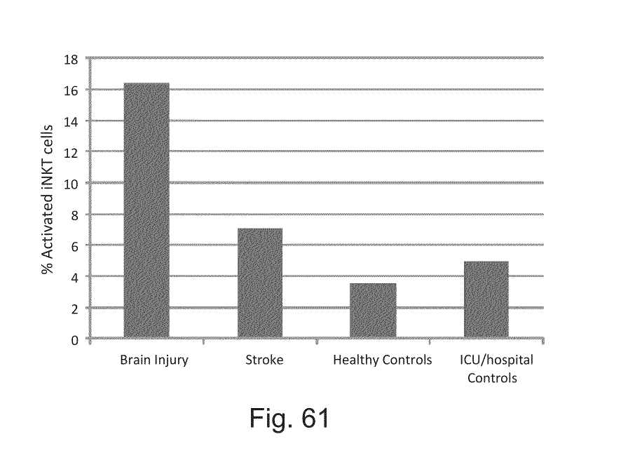

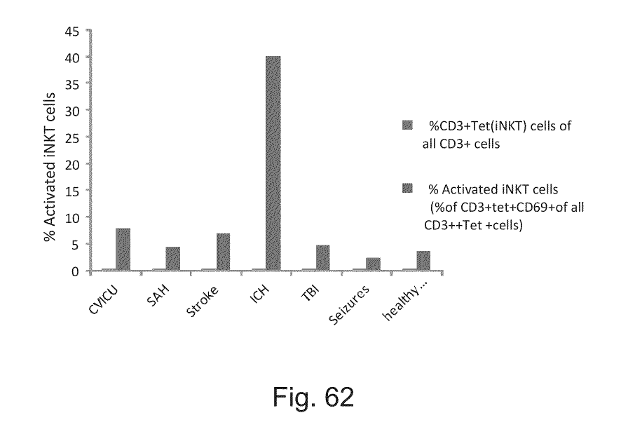

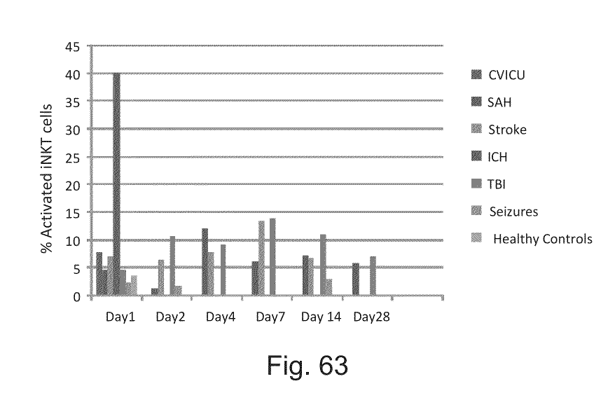

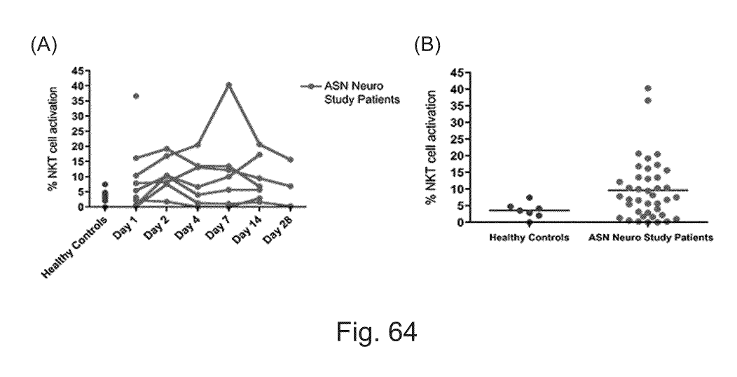

The data in FIG. 61 shows that the percentages of activated iNKT cells increased significantly in human subjects within the first 24 hours after the occurrence of a traumatic brain injury or a stroke compared to healthy control subjects. However, it appears that maximum levels iNKT cell activiation from brain-injured patients is with the ICH group, i.e., patients that experienced intracerebral hemorrhage (FIG. 62). It was noted that this elevated level of iNKT cell activity disappeared within 24 hours (FIG. 63). However, the production of activated iNKT cells remained significantly elevated in brain trauma patients, relative to the healthy controls, over the 28-day observation period (FIGS. 64A, 64B).

1-35. (canceled)

36. An α-galacytosylceramide compound having a chemical structure shown in Formula 3,

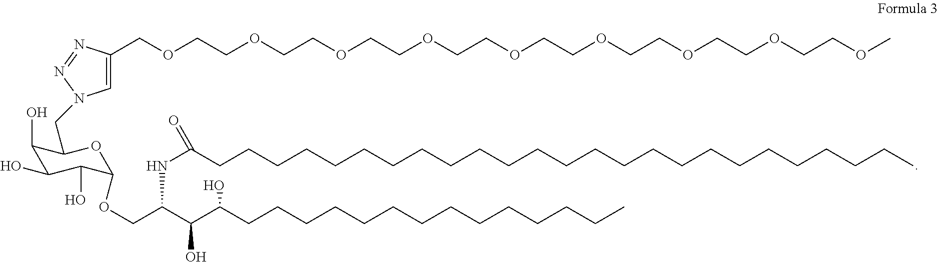 [SMILES: CCCCCCCCCCCCCCCCCCCCCCCCCC(=O)N[C@@H](COC1OC(Cn2cc(COCCOCCOCCOCCOCCOCCOCCOCCOC)nn2)C(O)C(O)C1O)[C@H](O)[C@H](O)CCCCCCCCCCCCCC]

37. An antimicrobial composition for intravenous administration to a subject who has experienced a brain trauma, the antimicrobial composition comprising:
the α-galacytosylceramide compound according to claim 36; and
one or more pharmaceutically acceptable excipients.

38. Use of the α-galacytosylceramide compound according to claim 36, for manufacture of an intravenous antibiotic medicament for prophylactic administration to a subject who has experienced a brain trauma, to reduce a potential for an occurrence in the subject of a post-trauma microbial infection.

39. A method for reducing the risk of a microbial infection in a subject who has experienced a brain trauma, the method comprising a step of administering to the subject as soon as possible after occurrence of the brain trauma, an effective amount of an intravenous dosage of the antimicrobial composition of claim 37.

40. The method of claim 39, wherein a second intravenous dosage is administered about 1 hour to about 96 hours after administration of the first intravenous dosage.

41. The method of claim 39, wherein a second intravenous dosage is administered about 2 hours to about 72 hours after administration of the first intravenous dosage.

42. The method of claim 39, wherein a second intravenous dosage is administered about 3 hours to about 48 hours after administration of the first intravenous dosage.

43. The method of claim 39, wherein a plurality of regularly scheduled intravenous dosages are administered, said administration of the plurality of intravenous dosages commencing about 4 hours to within 24 hours after administration of the first intravenous dosage.

44. The method of claim 39, additionally comprising the step of administering an oral dosage of an antibiotic composition, said administration of the plurality of oral dosages commencing about 24 hours to about 168 hours after administration of the first intravenous dosage.

45. The method of claim 39, wherein the brain trauma is a consequence of a stroke event.

46. The method of claim 45, wherein the stroke event is an ischemic stroke event.

47. The method of claim 39, wherein the brain trauma is a consequence of a physical injury to the subject's head.
